SEQUENCE LISTING (3 sequences)

SEQ ID NO: 1 - protein, 159 aa, Clostridium thermocellum
PVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 2 - DNA, 477 bp, Clostridium thermocellum
  source 1..477: mol_type = genomic DNA
ccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaaccc

SEQ ID NO: 3 - DNA, 97 bp, synthetic construct
  source 1..97: mol_type = other DNA
cgacgtcgctcgaatgccgggctcgcgttgccgagggggtgggtttgggtcaccactggcgtaggaagccaagggtgtggtgtgcagcgccgagcta

(19) United States
(12) Patent Application Publication
(10) Pub. No.: US 2024/0286127 A1
(43) Pub. Date: Aug. 29, 2024

(54) AN APPARATUS FOR A CELLULOSE-BASED VERTICAL FLOW ASSAY AND RELATED METHOD

(71) Applicants: Massachusetts Institute of Technology, Cambridge, MA (US); Nanyang Technological University, Singapore (SG)

(72) Inventors: Sing Mei Lim, Singapore (SG); Hoi Lok Cheng, Singapore (SG); Patthara Kongsuphol, Singapore (SG); Huan Jia, Singapore (SG); Say Yong Ng, Singapore (SG); Hadley Sikes Johnson, Cambridge, MA (US); Peter Preiser, Singapore (SG); Megan Earley McBee, Singapore (SG)

(21) Appl. No.: 18/682,662

(22) PCT Filed: Aug. 15, 2022
(86) PCT No.: PCT/SG2022/050579

§ 371 Date: Feb. 9, 2024

(30) Foreign Application Priority Data
Aug. 17, 2021 (SG) 10202109005S

(51) Int. Cl. B01L 3/00; G01N 33/58

(52) U.S. Cl. CPC B01L 3/5023; G01N 33/58; B01L 2300/045; B01L 2300/069; B01L 2300/0887; B01L 2300/12

(57) ABSTRACT

There is provided an apparatus for a cellulose-based vertical flow assay, the apparatus comprising: a holder for supporting a cellulose substrate having a plurality of test zones thereon, the holder having a base plate and engaging members; and a lid for reversibly engaging with the engaging members of the holder such that when engaged, the lid is disposed over the base plate and a gap exists between the lid and the base plate, wherein the lid comprises a plurality of openings for allowing access to the test zones on the cellulose substrate. Also provided are related methods.

### TECHNICAL FIELD

The present disclosure relates broadly to an apparatus for a cellulose-based vertical flow assay and related method.

### BACKGROUND

Various different types of assays that are based on the principle of antigen-antibody interaction on a membrane are present. These include dot immunoassay, dot immunoblot and paper-based enzyme-linked immunosorbent assay (ELISA).

In conventional dot immunoassay, antigens are spotted on hydrophobic nitrocellulose or polyvinylidenedifluoride (PVDF) membrane surface and then exposed by dipping the membrane to antibody-containing solutions. Subsequently, the membrane is transferred to reporter-conjugated antibody for signal detection. Likewise, conventional dot immunoblot (dot blot/western blot analysis) applies similar principles as dot immunoassay. In dot immunoblot, samples are dispensed into well plates with nitrocellulose membrane bottom. The samples are then treated with blocking reagent, primary and secondary antibody like a traditional western blot. Quantification of the signal can also be achieved using plate reader. However, the workflow for both dot immunoassay and dot immunoblot includes long blocking time, multiple washing steps, and long antibody incubation which can easily range from 1-3 hours depending on the setup. Furthermore, the entire assay may take about 1-2 days to complete.

In conventional paper-based ELISA, the overall assay time may be substantially shorter than that of the dot immunoblot and dot immunoassay. The assay is performed on a paper plate spotted with arrays of round micro-zones. During sample application, the test paper has to be suspended to prevent solutions from wicking through to facilitate direct adsorption of antibody onto the nitrocellulose membrane. Later, functionalization of nitrocellulose membrane with epoxide or recombinant nitrocellulose anchoring protein is adopted to increase antibody adsorption onto the membrane surface. Although paper-based ELISA may provide a quicker turnaround time as compared to dot immunoblot and dot immunoassay, it requires additional functionalization steps to be performed on the nitrocellulose membrane to ensure sufficient antibody absorption onto the membrane.

In particular, all of the aforementioned methods use mainly nitrocellulose or PVDF membrane, which can be considered as specialised membranes, as the substrate for antibody/antigen adsorption. Such specialised membranes can be relatively costly, especially when employed on a large scale for high throughput screening, and they may also require additional processing steps, which further increase the overall cost of the assays. Moreover, some of the methods are limited to antibody-antigen interaction and may not be extended to non-antibody ligand based interaction.

Thus, there is a need to provide an alternative apparatus and/or method for performing assays that address or at least ameliorate one or more of the above-mentioned problems. In particular, there is a need for an alternative apparatus and/or method that are compatible with less/non-specialized membranes and yet allow for high throughput.

### SUMMARY

In one aspect, there is provided an apparatus for a cellulose-based vertical flow assay, the apparatus comprising: a holder for supporting a cellulose substrate having a plurality of test zones thereon, the holder having a base plate and engaging members; and a lid for reversibly engaging with the engaging members of the holder such that when engaged, the lid is disposed over the base plate and a gap exists between the lid and the base plate, wherein the lid comprises a plurality of openings for allowing access to the test zones on the cellulose substrate.

In one embodiment, the apparatus further comprises one or more layers of cellulose substrate disposed over the base plate of the holder, and the cellulose substrate having a plurality of test zones thereon.

In one embodiment, the apparatus further comprises one or more layers of an absorbent material disposed over the base plate. Optionally, in one embodiment, the absorbent material is disposed between a cellulose substrate and the base plate.

In one embodiment, the gap is capable of accommodating a plurality of layers of a cellulose substrate and a plurality of layers of an absorbent material simultaneously.

In one embodiment, the engaging members of the holder comprise slots for slidably engaging the lid.

In one embodiment, the plurality of openings of the lid each aligns to a respective test zone of the cellulose substrate.

In one embodiment, the test zones of the cellulose substrate are relatively more permeable to a liquid sample than areas of the cellulose substrate that are outside the test zones. Optionally, in one embodiment, the areas of the cellulose substrate that are outside the test zones are coated with a layer that is substantially impermeable to a liquid sample.

In one embodiment, the lid comprises no less than 60 openings for allowing access to the test zones on the cellulose substrate.

In one embodiment, the one or more layers of cellulose substrate comprises a plurality of layers of cellulose substrate and the diameter of the test zones of a cellulose substrate selected from the plurality of layers of cellulose substrate is different from the diameter of the test zones of another cellulose substrate selected from the plurality of layers of cellulose substrate.

In one aspect, there is provided a method of detecting an analyte in a sample, the method comprising: providing an apparatus comprising, a holder having a base plate and engaging members; one or more layers of cellulose substrate disposed over the base plate, the cellulose substrate having a plurality of test zones thereon; one or more layers of an absorbent material disposed between the base plate and the cellulose substrate; and a lid reversibly engaged with the engaging members of the holder such that the lid is disposed over the base plate, the cellulose substrate and the absorbent material, and wherein the lid comprises a plurality of openings for allowing access to the test zones on the cellulose substrate; applying the sample to one or more test zones of the cellulose substrate through the one or more openings of the lid to allow said analyte, if present in said sample, to be captured onto said one or more test zones of the cellulose substrate by a capture agent that is immobilized on the one or more test zones, wherein said capture agent is:
(i) incubated with said sample prior to the applying step; and/or
(ii) immobilised on the one more or more test zones of the cellulose substrate prior to the applying step; and determining a presence or absence of said analyte captured on the one or more test zones of said cellulose substrate by said capture agent.

In one embodiment, determining a presence or absence of said analyte captured on the one or more test zones by said capture agent comprises detecting a signal effected by a reporter agent on the one or more test zones, wherein said reporter agent:
(i) comprises an analyte binder and said reporter agent is contacted with the sample to allow said reporter agent to bind to said analyte, if present, in said sample; or
(ii) comprises a competing binder having affinity for said capture agent and said reporter agent is contacted with said capture agent to allow said reporter agent to bind to said capture agent.

In one embodiment, the method further comprises, after the applying step but prior to the determining step, washing the one or more test zones with a washing reagent to remove free molecules from the cellulose substrate.

In one embodiment, the method further comprises blocking the cellulose substrate with a blocking agent.

In one embodiment, where said reporter agent comprises an analyte binder, the method comprises incubating the reporter agent and the capture agent with said sample prior to the applying step and said analyte when present, is bound to the reporter agent to form part of a reporter agent-analyte-capture agent complex prior to the applying step.

In one embodiment, where said reporter agent comprises an analyte binder, the method comprises incubating the reporter agent with said sample and immobilizing said capture agent on the one or more test zones of said cellulose substrate prior to the applying step and said analyte when present, is bound to the reporter agent to form a reporter agent-analyte complex prior to the applying step.

In one embodiment, where said reporter agent comprises a competing binder, the method comprises incubating said capture agent with said reporter agent and/or said sample prior to the applying step to form a reporter agent-capture agent complex and/or an analyte-capture agent complex prior to the applying step.

In one embodiment, where said reporter agent comprises a competing binder, the method comprises immobilizing said capture agent on the one or more test zones of said cellulose substrate prior to the applying step and dispensing the reporter agent on the one or more test zones after the applying step.

In one embodiment, the applying step comprises allowing the sample to flow through the one or more test zones of the cellulose substrate to the absorbent material.

In one embodiment, the method further comprises adding a reagent capable of producing a detectable signal upon reaction with the reporter agent after the applying step.

In one embodiment, the capture agent has affinity for cellulose. Optionally, in one embodiment, the capture agent comprises a cellulose binding domain.

### Definitions

The term “substrate” as used herein includes any carrier or matrix upon which interaction between two or more substances (e.g., protein-protein interaction, antibody-antigen interaction, protein-antigen interaction etc.) can take place.

The terms “contacting”, “contacted” and the like as used herein in relation to two or more substances broadly include conditions which allow contact or interaction between the two or more substances whether in solution or in solid phase. For example, contacting two or more substances may comprise mixing together the two or more substances in solution or adding the two or more substances to the same substrate (for example, adding the two or more substances to the same test zone). The terms do not necessarily require actual physical contact between the two or more substances. The terms “incubating”, “incubated” and the like as used herein in relation to two or more substances means contacting two or more substances for a sufficient duration of time to allow interaction between the two or more substances to take place. No specific temperature requirements are implied by the terms “incubating”, “incubated” and the like unless otherwise indicated.

The terms “bind,” “binding,”, “bound” and the like as used herein broadly include to any type of immobilization, formation of a complex, fixation, or attachment between two or more substances, between a substance and one or more surfaces, or a combination thereof, regardless of the mechanism or mechanisms of attachment involved. Examples of mechanisms include, but are not limited to, formation of complexes (e.g., protein-protein complex, antibody-antigen complex, non-antibody protein-antigen complex etc.), covalent bonding, ionic bonding, electrostatic forces, hydrogen bonding, van der Walls attraction, hydrophobic effect, absorption and adsorption.

The term “affinity” as used herein refers to an attraction or non-random interaction between two molecules or parts thereof, for example, between cellulose and cellulose-binding domain.

The term “micro” when used as a unit prefix (e.g., 1 micro (μ) generally denotes a factor of 10⁻⁶ When used in relation to dimensions, the term is to be interpreted broadly to include dimensions from about 1 micron to about 1000 microns.

The term “nano” when used as a unit prefix, (e.g., 1 nano (n)) generally denotes a factor of 10⁻⁹ When used in relation to dimensions, the term is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. In some examples, the term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle. In some examples, the term “size” when used to refer to the particle broadly refers to the weight of the particle. For example, the “size” of a protein may be described using its molecular mass in kilodaltons (kDa) unit.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship.

For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of an apparatus for a cellulose-based vertical flow assay and methods of detecting an analyte in a sample using said apparatus/device are disclosed hereinafter.

### Apparatus/Device

In various embodiments, there is provided an apparatus/device for a cellulose-based vertical flow assay, the apparatus comprising a holder for supporting a substrate (e.g., a cellulose substrate) having a plurality of test zones thereon, the holder having a base plate and engaging members; and a lid for reversibly engaging with the engaging members of the holder such that when engaged, the lid is disposed over the base plate and a gap exists between the lid and the base plate, wherein the lid comprises a plurality of openings for allowing access to the test zones on the cellulose substrate. In various embodiments, the holder is adapted to support a cellulose substrate. The cellulose substrate may comprise, at least in part, cellulose and/or other polysaccharide equivalent of cellulose e.g., to which a cellulose-binding domain can bind. An example of a cellulose substrate is cellulose paper. A cellulose substrate further includes any functionalised cellulose substrates, such as cellulose substrates which surface are functionalised with e.g., nanoparticles. Advantageously, cellulose materials/membranes/papers are lower in cost and does not require much processing, if any, as compared to nitrocellulose membranes and polyvinylidene fluoride (PVDF) membranes.

Advantageously, embodiments of the apparatus are simple and adapted to be used with cost-effective cellulose paper. When coupled with a modified workflow, embodiments of the apparatus can function as a high-throughput cellulose-based pull-down assay to effectively detect/analyse protein-protein interactions in a cost-effective manner. Accordingly, embodiments of the apparatus and its associated workflow may be used in rapid diagnostic testing in a point-of-care setting. Embodiments of the apparatus and its associated workflow may also enable sample screening of a large cohort.

Embodiments of the apparatus and methods using said apparatus may apply the principles of antibody/antigen interactions to perform the assays, although they are not limited/restricted as such. For example, embodiments of the apparatus and methods using said apparatus may also rely on non-antibody ligand based interaction. Such flexibility allow for a wide array of assay possibilities. In various embodiments, the apparatus and related methods are suitable for indirect ELISA, sandwich ELISA and competitive ELISA experiments. Accordingly, in some embodiments, the apparatus and related methods may be considered to pertain to an improved/alternative/modified ELISA assay. For example, embodiments of the apparatus are specially designed for performing vertical flow assays and are unlike conventional microtiter ELISA plates. For example, embodiments of the apparatus and its associated methods keep similar pressure distribution across a plate, thus allowing a vertical flow feature to simplify washing step by absorption across a large number of samples. This may significantly reduce washing and handling time as compared to microtiter ELISA plates.

In various embodiments, as the lid of apparatus may be disengaged/detached from the apparatus, easy access is provided to insert, remove and/or replace one or more cellulose substrates from the apparatus. Furthermore, the plurality of openings on the lid also allow for easy access to the test zones of a cellulose substrate disposed in the holder (e.g., to provide/apply the test sample and/or other reagents to the test zones of the cellulose substrate) when the lid is fully engaged over the holder.

In various embodiments, the apparatus may be provided in a fully assembled form, partly assembled form or a disassembled form. For example, when the apparatus is in a fully assembled form, the lid may be fully engaged with the holder. Alternatively, when the apparatus is in the unassembled/disassembled form, the lid may be fully disengaged/detached/separated from the holder. It will be appreciated that in various embodiments, engaging the lid with the engaging members of the holder allows the lid to be substantially supported and suspended over the base plate.

In various embodiments, the lid is slidably engaged with the engaging members of the holder. Advantageously, this allows for easy engagement and disengagement of the lid with the holder. Even more advantageously, various embodiments of the sliding mechanism allow for substantially equal distribution of pressure across the holder and base plate upon assembly for consistent flow rate of the samples through the substrate. In various embodiments, the engaging members of the holder comprise slots for slidably engaging the lid. For example, the slots allow the lid to slide and be slotted therein to be in an engaged configuration. The slots may be in the form of indents or grooves on a surface of each of the engaging member. The slots may be through slots, wherein at least one end of each of the slots is a through opening. Insertion and removal of the lid may be allowed via the through opening. In various embodiments, both ends of the slots may also comprise through openings so that insertion and removal of the lid from the slots may be carried out at either end. Thus, in various embodiments, the engaging members allow the lid to be completely removed from the holder. Accordingly, in various embodiments, the lid is configured to detachably engage with the engaging members. In various embodiments, the engaging members extend substantially vertically from the base plate. Thus, in various embodiments, when the lid is engaged with the substantially vertical engaging members, the lid is substantially parallel to the base plate. In various embodiments, the vertical engaging members provide substantially balanced support surfaces on their lateral edges for the lid. The engaging members and/or the lids may also have detents from preventing the lid to be removed from the engaging members e.g., from one end or both ends of the engaging members. For example, if both ends of the engaging members have detents, the lid may be prevented from being completely removed from the engaging members. In one example, the engaging members may be coupled to a detent in the form of a wall on a distal edge of the engaging members to block the lid from sliding out from a side of the holder.

It will be appreciated that other types of engagement methods or mechanisms may also be used so long as they allow for reversible engagement of the holder and the lid. For example, hinge/swivel mechanisms, clip mechanisms, catch mechanisms, latch mechanisms, snap-on mechanisms or the like, or combinations thereof may also be used.

In various embodiments, when in the engaged configuration, a gap or a space exists between the lid and the base plate. The gap or space is able to or is dimensioned to accommodate one or more layers of cellulose substrate and/or one or more layers of absorbent material. Accordingly, in various embodiments, the apparatus comprises one or more layers of cellulose substrate and optionally one or more layers of absorbent material disposed over the base plate of the holder. The gap or space may also be such that when the desired layers of cellulose substrate and optionally one or more layers of absorbent material are disposed over the base plate of the holder, the top-most layer of cellulose substrate contacts the lid with no appreciable empty space existing between the lid and the top-most layer of the cellulose substrate. For example, the layers of cellulose substrate and optionally one or more layers of absorbent material that are disposed over the base plate of the holder exert a pushing force upwards towards the lid, allowing the top-most layer of cellulose substrate to contact the lid.

The cellulose substrate may have a plurality of test zones thereon. In various embodiments, the test zone is substantially circular in shape. In one embodiment, the test zone comprises a round microzone. It will be appreciated that the shape of the test zones is not particularly limited to a circular/round shape and the test zones may also be of other shapes e.g., square, rectangle, oval, ellipsoid, triangle, parallelogram or the like or combinations thereof. In such embodiments, the shape of the openings on the lid may be configured to correspond to the shape of the test zones. The shapes of the plurality of test zones on the substrate may be the same or different. In one embodiment, the shapes of the plurality of test zones on the substrate are the same. When a plurality of substrates is used, the shape and/or size of the test zones on one substrate may be the same or different from the shape and/or size of the test zones on another substrate. When a plurality of substrates is used, the shape and/or size of the test zones on/within each substrate may be the same or different. In one embodiment, the shape of the test zones and/or size on every substrate are the same.

In various embodiments, the sizes/areas/diameters/widths of the test zones are substantially similar to a size/area/diameter/width of the bottom of a well in a well plate. In various embodiments, the sizes/areas/diameters/widths of the test zones are substantially similar to a size/area/diameter/width of the bottom of a well in a 96-well plate. It will be appreciated that the sizes/area/diameters of the test zones are not particularly limited and may be varied according to the requirements of the assay. In various embodiments, the diameter/width of the test zones are from about 3 mm to about 10 mm, or from about 5 mm to about 9 mm. In one embodiment, the diameter/width of the test zones are about 7 mm. When a plurality of substrates is used, the size/area/diameter/width of the test zones on one substrate may be the same or different from the size/area/diameter/width of the test zones on another substrate. When a plurality of substrates is used, the size/area/diameter/width of the test zones on each substrate may be the same or different. In one embodiment, the size/area/diameter/width of the test zones on every substrate are the same. In one embodiment, the size/area/diameter/width of the test zones on every substrate are different. In one embodiment, where the one or more layers of cellulose substrate comprises a plurality of layers of cellulose substrate, the diameter of the test zones of a cellulose substrate selected from the plurality of layers of cellulose substrate is different from the diameter of the test zones of another cellulose substrate selected from the plurality of layers of cellulose substrate

In various embodiments, the number of test zones present on the substrate are no less than about 60, no less than about 65, no less than about 70, no less than about 75, no less than about 80, no less than about 85, no less than about 90, no less than about 95 or no less than about 96. The number of test zones can be increased or decreased depending on the requirements of the assay. Advantageously, embodiments of the apparatus comprising the substrate are suitable for high throughput screening.

In various embodiments, the test zones of the cellulose substrate are relatively more permeable to a liquid sample than areas of the cellulose substrate that are outside the test zones. For example, the areas of the cellulose substrate that are outside the test zones are coated with a layer that is substantially impermeable to a liquid sample. Advantageously, the relative impermeability of areas outside the test zones may allow for the sample to concentrate in the test zones, subsequently allowing results of the assays to be focused on the test zones. Cross-contamination between test zones may also be minimised/prevented. The relative impermeability of areas outside the test zones may also substantially prevent adsorption of molecules in these areas. Accordingly, the washing buffer may be used to easily wash unbounded molecules from these areas. This may advantageously improve the specificity of the assay by reducing the likelihood of non-specific binding in the areas outside the test zones, which may interfere with the accuracy of the results read-out or detection.

In various embodiments, the layer that is substantially impermeable to a liquid sample comprises a hydrophobic layer. Accordingly, in various embodiments, the cellulose substrate comprises a waxed cellulose substrate (e.g., waxed cellulose paper). In various embodiments, the test zones of the cellulose substrate are not waxed. The waxed cellulose substrate may be obtained by applying printing techniques such as wax printing and/or screen printing. For example, a printer using hydrophobic polymer solutions and/or wax ink may be used to create hydrophobic patterns on cellulose paper (e.g., filter paper) to create defined hydrophobic barriers around the unprinted zones which can eventually serve as hydrophilic test zones. Examples of suitable wax ink include, but are not limited to, Fuji Xerox Colorqube Solid ink and polystyrene ink (alkyl ketyl dimer in p-xylene).

The apparatus may further comprise one or more layers of an absorbent material disposed over the base plate. Advantageously, the absorbent material may serve to remove excess unbound molecules and washing buffer via capillary force and gravity. The absorbent material may also provide a wicking/pulling force to enhance the flow of the sample and subsequently the completion of the assay. The absorbent material may be disposed between a cellulose substrate and the base plate, that is the absorbent material may be sandwiched between the cellulose substrate and the base plate such that in a vertical flow assay, the sample/fluid will pass through the cellulose substrate before reaching the absorbent material. In one embodiment, the absorbent material comprises Whatman GB005 blotting paper. Nonetheless, it will be appreciated that other types of absorbent material or blotting paper may be used as long as they may adequately remove excess unbound molecules and washing buffer during the assays. For example, other porous material (not limited to cellulose materials) may also be used e.g., ReliaFlow 440 from Ahlstrom-Munksjô made of a mix of cotton and glass fibres.

In various embodiments, the gap or space is capable of accommodating different varying thicknesses of substrates (e.g., cellulose substrates) and absorbent material. For example, the gap or space may be capable of accommodating a plurality of layers of a cellulose substrate and a plurality of layers of an absorbent material simultaneously. Advantageously, a user may be able to vary a type and/or thickness and/or a number of the layers of cellulose substrate and the absorbent material to adjust the volume of sample/reagents allowed to be dispensed onto the test zones, to tweak the rate of flow of sample/reagents, to adjust the amount of sample/reagent/washing buffer to be adsorbed onto the cellulose substrate, and/or to ensure that no excess fluid flows out of the apparatus. For example, when the absorbent material is Whatman GB005 blotting paper, the total volume of sample and reagents allowed to be dispensed onto the test zones should be no more than about 200 μL. The total volume of sample and reagents allowed to be dispensed onto the test zones may vary when other types of absorbent materials/blotting papers are used. Optimization may be carried out to determine the appropriate total volume of sample and reagents allowed to be dispensed onto the test zones.

In various embodiments, the height of the gap or space is from about 2 mm to about 7 mm or from about 3 mm to about 5 mm. In one example, the height of the gap or space is from about 3.3 mm to about 4.8 mm.

In various embodiments, the apparatus comprises at least about 1 layer of absorbent material, at least about 2 layers of absorbent material, at least about 3 layers of absorbent material or at least about 4 layers of absorbent material disposed over the base/bottom plate. Depending on the thickness of the absorbent material and the height of the gap or space in the apparatus, the number of layers of absorbent material that would fit the gap may differ. In one example, where the absorbent material used is Whatman GB005 blotting paper having a thickness of about 1.5 mm and the height of the gap or space is from about 3.3 mm to about 4.8 mm, the gap or space is fitted by 2 or 3 layers of Whatman GB005 blotting papers together with 3 layers of cellulose substrate in the form of Whatman No. 1 filter papers.

In various embodiments, the apparatus comprises at least about 1 layer of cellulose substrate, at least about 2 layers of cellulose substrate, at least about 3 layers of cellulose substrate, at least about 4 layers of cellulose substrate or at least about 5 layers of cellulose substrate disposed over the base/bottom plate. Depending on the height of the gap or space in the apparatus, it may also be possible to fit more layers of cellulose substrate in the apparatus.

In one embodiment, the apparatus comprises 3 layers of cellulose substrates and 3 layers of absorbent material are used. Nonetheless, it will be appreciated that in various embodiments, the number of layers of cellulose substrates and the number of layers of absorbent used may be the same or different.

In various embodiments, the number of test zones in a layer of cellulose substrate are no more than the number of test zones in the layer immediately below it. In various embodiments, the number of test zones in each layer of cellulose substrate are the same.

The test zones in each layer may also be substantially aligned to the corresponding test zones of the other layers. In various embodiments, where the test zones in one layer are of same or different sizes from the test zones of another layer, the center of each test zone in one layer and the center of each respective corresponding test zone in the other layer may be substantially aligned (e.g., a line that is passing through both centers is substantially perpendicular to the surface of the cellulose substrate on which the test zones reside and/or the bottom plate). For example, if the test zones in all layers are circular in shape, the test zones in each layer may be aligned such that the test zones on are substantially concentric to corresponding test zones in another layer. In various embodiments, the centers of the corresponding test zones in all of the different layers are substantially aligned to one another (e.g., substantially concentric to one another).

The flow rate of the sample (and/or any other reagents added to the test zones) passing through the cellulose substrates may be optimized for signal generation. Without being bound by theory, it is believed that the flow rate of a sample (and/or any other reagents) depends on two factors: (i) the binding affinity of protein of interest/analyte in the sample with its capture agent and (ii) the viscosity of the sample. This can be adjusted by (a) changing the test zone sizes across the cellulose substrate layers to alter the capillary effect and gravity flow and/or (b) changing the viscosity of the sample (and/or any other reagents added to the test zones).

In various embodiments, when there is a plurality of layers of cellulose substrate present, the test zones in each layer of substrate may be varied to adjust the flow rate of the sample through the test zones. For instance, the size of the test zones in each layer of cellulose substrate may be different. In such a situation, due to the difference in the sizes of the overlapping hydrophilic areas of the test zones between the layers (e.g., between 1^{st} and 2^{nd} layers and between 2^{nd} and 3^{rd} layers etc), the flow rate of the sample through the cellulose substrates may be adjusted. To illustrate this point, take for example an embodiment where the test zones in each layer are substantially circular and the center of these test zones are substantially aligned, the diameter of the test zones in each layer may be altered as follows: Option (i): 1^{st} layer (or top most layer)—5 mm, 2^{nd} layer—5 mm, 3^{rd} layer—5 mm; Option (ii)₁ₛt layer—5 mm, 2^{nd} layer—4 mm, 3^{rd} layer—4 mm Option (iii): 1^{st} layer—5 mm, 2^{nd} layer—4 mm, 3^{rd} layer—3 mm Option (iv): 1^{st} layer—5 mm, 2^{nd} layer—3 mm, 3^{rd} layer—3 mm. In this illustration, the sample flow rate decreased in the following order: Option (i), Option (ii), Option (iii) and Option (iv). Thus, a flow rate of a liquid sample can be adjusted by varying the diameters of test zones across the substrates depending on the sample viscosity.

It will be appreciated that instead of only relying on the difference in size of the test zones between layers, other manners of varying the test zones in each layer may also be useful to vary the sample flow rate across the layers. For example, varying the alignment between the corresponding test zones of each layer of the cellulose substrate (e.g. such that the centers of the respective test zones between layers may be offset from each other or misaligned) may in turn result in the overlapping hydrophilic areas of the test zones between the layers (e.g. between 1^{st} and 2^{nd} layers and between 2^{nd} and 3^{rd} layers etc) being varied, thereby allowing the flow rate of the sample through the cellulose substrates to be adjusted.

In various embodiments, the test zones of the cellulose substrate may further comprise one or more capture agents (e.g., capture proteins) immobilized thereon. These capture agents (e.g., capture proteins) may be capable of coupling to molecules or analytes of interest.

In various embodiments, the lid comprises no less than about 60, no less than about 65, no less than about 70, no less than about 75, no less than about 80, no less than about 85, no less than about 90, no less than about 95, or no less than about 96 openings for allowing access to the test zones on the cellulose substrate. In various embodiments, the number of openings in the lid is no more than the number of test zones on the cellulose substrate that is nearest to the lid (for example, the top-most layer of cellulose substrate, that is the layer which the sample first passes through during an assay).

In various embodiments, the number of openings matches the number of test zones on the cellulose substrate. For example, the number of openings in the lid matches the number of test zones on at least the top-most layer of cellulose substrate. In some embodiments, the number of openings in the lid matches the number of test zones in all layers of the cellulose substrate.

The openings of the lid may be of any suitable shape, such as circle, square, rectangle, oval, ellipsoid, triangle, parallelogram or the like or combinations thereof. The openings of the lid may have substantially the same shape or have a different shape from the test zones of the cellulose substrate. In various embodiments, the area of each opening may be substantially no larger than the area of a corresponding test zone on the cellulose substrate (e.g., the top-most layer of the cellulose substrate). In various embodiments, the area of each opening may be substantially the same as the area of a corresponding test zone on the cellulose substrate (e.g., the top-most layer of the cellulose substrate).

The openings in the lid may also be substantially aligned to the respective corresponding test zones of the cellulose substrate. In some embodiments, the plurality of openings of the lid each aligns to a respective test zone of the cellulose substrate. In various embodiments, the center of each opening of the lid substantially matches or is substantially aligned with the center of a corresponding test zone of the cellulose substrate (e.g., at least the top-most layer of cellulose substrate). That is, a line that is passing through both an opening center and a corresponding test zone center is substantially perpendicular to the surface of the cellulose substrate on which the test zones reside and/or the bottom plate. For example, if openings of the lid and the test zones are circular in shape, the openings of the lid may be aligned such that the corresponding test zones on the cellulose substrate (at least the top-most layer) are substantially concentric one another. In various embodiments, the centers of the openings and the centers of the corresponding test zones in all of the different layers are substantially aligned to one another (e.g., substantially concentric to one another).

In various embodiments, when the apparatus is fully assembled with the cellulose substrate present, each opening of the lid together with the cellulose substrate form a well. In various embodiments, therefore, the openings of the lid and the cellulose substrate form a plurality of wells, with the cellulose substrate forming the bases of the wells. The openings of the lid may be separated from one another by walls, which may be part of the lid. For example, each opening may be in a form of a channel which is separated from adjacent openings (also in the form of channels) by walls. Therefore, these walls may also correspond to the walls of the wells formed by the openings of the lid and the cellulose substrate.

The walls may be substantially opaque to minimize, substantially avoid or substantially prevent signal interference from signals emitted from adjacent wells (especially from the cellulose surface of the other wells) during the detection phase (e.g., fluorescence detection) of an assay.

The material used to make the lid, the holder, the engaging members and the base plate of the apparatus may be independently selected from, but not limited to, polystyrene, polycarbonate, polylactic acid, acrylonitrile butadiene styrene, PMMA, glass and any other polymers or 3D printing resins.

The apparatus may be dimensioned or adapted to fit a standard plate reader that is commercially available in the market. The apparatus may also be dimensioned or adapted to be compatible with a multi-channel pipette and plate reader, making it convenient to be adopted for lab-usage. In various embodiments, the width of the apparatus is from about 70 mm to about 100 mm or from about 80 mm to about 90 mm. In various embodiments, the length of the apparatus is from about 100 mm to about 130 mm, or from about 110 mm to about 120 mm. In various embodiment, the height of the apparatus is from about 8 mm to about 20 mm or from about 10 mm to about 15 mm. In various embodiments, the width×length×height of the apparatus is about 85 mm×118 mm×12 mm. In one example, the width×length×height of the apparatus is 85.5 mm×118.27 mm×12.52 mm.

### Method

There is also provided a method of detecting an analyte in a sample using the apparatus disclosed herein, for example, a fully assembled form of the apparatus with a cellulose substrate and absorbent material disposed over the base plate. Therefore, the method may comprise providing an apparatus comprising, a holder having a base plate and engaging members; one or more layers of cellulose substrate disposed over the base plate, the cellulose substrate having a plurality of test zones thereon; one or more layers of an absorbent material disposed between the base plate and the cellulose substrate; and a lid reversibly engaged with the engaging members of the holder such that the lid is disposed over the base plate, the cellulose substrate and the absorbent material, and wherein the lid comprises a plurality of openings for allowing access to the test zones on the cellulose substrate. The method may also comprise baking the cellulose substrate prior to the step of providing the apparatus. Baking may be carried out at a temperature of from about 100° C. to about 200° C. In one embodiment, baking is carried out at a temperature of 150° C. The duration of the baking may be from about 30 seconds to about 2 minutes. In one embodiment, the duration of the baking is about 1 minute.

The method may further comprise applying the sample to one or more test zones of the cellulose substrate through the one or more openings of the lid to allow said analyte, if present in said sample, to be captured onto said one or more test zones of the cellulose substrate by a capture agent that is immobilized on the one or more test zones, wherein said capture agent is:
(i) incubated with said sample prior to the applying step; and/or
(ii) immobilised on the one or more test zones of the cellulose substrate prior to the applying step; and determining a presence or absence of said analyte captured on the one or more test zones of said cellulose substrate by said capture agent. Methods for determining whether an analyte is captured on a cellulose substrate that are known in the art may be suitably applied in the disclosed method. For example, a detectable reporter agent may be used to bind to the analyte or the capture agent on the cellulose substrate and the signal effected by the reporter agent may indicate the presence or absence of analyte on the cellulose substrate.

The capture agent may have affinity for cellulose. The capture agent may bind specifically to cellulose. The capture agent may comprise at least one of a functionalized entity configured to chemically couple to cellulose, a cellulose binding domain (CBD), or a cellulose binding/anchoring aptamer. An example of a cellulose binding/anchoring DNA aptamer is CELAPT 14 (SEQ ID NO. 3: 5′-CGACGTCGCTCGAATGCCGGGCTCGCGTTGCCGAGGGGGTGGGTTTGGG TCACCACTGGCGTAGGAAGCCAAGGGTGTGGTGTGCAGCGCCGAGCTA).

In some embodiments, the capture agent comprises an antibody (e.g., an antibody against the analyte) covalently immobilised on the surface of the cellulose substrate. In one embodiment, the capture agent comprises a CBD.

In various embodiments, the method further comprises, after the sample applying step but prior to the determining/detecting step, washing the one or more test zones with a washing reagent to remove free molecules from the cellulose substrate. Advantageously, this can improve the accuracy of the detection of signal effected by the reporter agent to determine the presence or absence of said analyte captured on said cellulose substrate by said capture agent. As unbounded and/or weakly bounded molecules (e.g., not containing the target analyte of interest) are removed, the likelihood of obtaining false positives (e.g., when the reporter agent is one that binds to an analyte) is also reduced.

The method may also further comprise, blocking the cellulose substrate with a blocking agent/buffer. The blocking step may be carried out prior to the sample applying step. For example, when the capture agent has been incubated with the sample prior to the sample applying step, the blocking step may be carried out prior to the sample applying step. For example, when the capture agent is immobilised on the one more or more test zones of the cellulose substrate prior to the sample applying step, the blocking step may be carried out after applying the capture agent to the test zones and before applying the sample. Advantageously, the blocking step may improve the specificity of an assay by reducing background interference and improving the signal-to-noise ratio. Examples of blocking agents/buffers include, but are not limited to, bovine serum albumin (BSA) e.g., at from about 1 to about 5% concentration, non-fat dry milk (NFDM) e.g., at from about 0.1 to about 3% concentration, fish gelatin, whole sera, polyethylene glycol (PEG), polyvinyl alcohol (PVA), and polyvinylpyrrolidone (PVP).

In various embodiments, the method further comprises incubating the capture agent with said sample prior to the applying step. Alternatively, in other embodiments, the method further comprises immobilizing said capture agent on the one or more test zones of said cellulose substrate prior to the applying step.

In various embodiments, the method is a vertical flow assay and the sample applying step comprises allowing the sample to flow through the one or more test zones of the cellulose substrate to the absorbent material.

Detecting an analyte may comprise detecting a presence, an absence, an amount/concentration, or a relative amount/concentration of an analyte. The detection may be qualitative, quantitative or semi quantitative. For example, an amount/concentration or a relative amount/concentration of an analyte can be determined by measuring an intensity of the signal or a rate of production of the signal effected by the reporter agent, and then comparing the intensity or rate with that effected by a control sample or a comparative sample containing a known amount of analytes. If the intensity or rate of the signal obtained from the sample is greater than that obtained from the control sample or the comparative sample in a non-competitive binding assay, this may be indicative that the sample contains a greater amount/concentration of the analyte than the control sample or the comparative sample. The amount/concentration of the analyte in the sample may be determined by calculating the ratio of the intensity or rate obtained from the sample to the intensity or rate obtained from the control sample or the comparative sample.

The method of detecting an analyte may also comprise detecting two or more analytes, or a plurality of analytes. For example, two or more different analytes, or a plurality of different analytes, may be detected by use of different reporter agents, or by capturing the different analytes onto different spatial regions on the cellulose substrate.

The term “analyte”, “analyte of interest” or “target analyte” as used herein broadly refers to a substance which is to be detected/measured. In various embodiments, an “analyte” not only includes any substance for which there exists a naturally occurring binding member specific to the analyte, but also includes any substance for which a binding member specific to the analyte can be prepared e.g., an affitin. Examples of analytes include, but are not limited to, prokaryotic or eukaryotic cells of any type, microorganisms, bacteria, pathogens, viruses, prions, organic compounds, lipids, carbohydrates, hormones, antibodies, antigen-binding proteins, peptides, amino acids, nucleic acids/polynucleotides such as deoxyribonucleic acid (DNA) and ribonucleic acids (RNA), steroids, vitamins, drugs (including those administered for therapeutic purposes as well as those administered for illicit purposes), drug intermediates, toxins, chemicals, pesticides, pollutants, metal, heavy metal, metal ions and metabolites, portions, fragments and extracts of the aforementioned materials and combinations thereof. In some embodiments, the analyte comprises a virus. In some embodiments, the analyte comprises a viral protein. In some embodiments, the analyte, or portion thereof, is antigenic or haptenic having at least one determinant site, or is a member of a binding pair.

In some embodiments, the analyte comprises a biological material. In some embodiments, the analyte comprises a natural material, for example, a naturally occurring biological material. In some embodiments, the analyte comprises a synthetic material, for example, pesticides.

The sample (or a test sample) may be a sample suspected of containing an analyte. A sample suspected of containing an analyte may refer to a sample for which the content of the analyte is unknown or unconfirmed. For example, a sample from a human suspected of having a disease (and therefore suspected of having the disease-associated analyte in his/her sample), but not known to have the disease, may constitute a sample suspected of containing an analyte. Embodiments of the method may also be used for confirmatory or further testing of a sample (e.g., to confirm the presence or absence of an analyte in the sample, or to further determine an amount/relative amount of the analyte in the sample) that has already been evaluated earlier by another method. Embodiments of the method also provide for a control sample that may be used to obtain an indication on whether the method is correctly performed. A “control sample” is distinguished from the “sample” or “test sample” in that the contents of the analyte (presence, absence etc.) in the “control sample” is known. A “control sample” includes both negative and positive control samples.

The sample may be obtained from any source. For example, the sample may be a biological sample, a pharmaceutical sample, an environmental sample, a food sample etc. In some embodiments, the sample comprises a biological sample. Examples of biological samples include, but are not limited to blood, serum, plasma, sputum, saliva, lavage fluid (e.g. bronchial lavage fluid, alveolar lavage fluid and bronchoalveolar lavage fluid), nasal fluid/swab/wash/aspirate, anterior nares fluid/swab/wash/aspirate, nasal mid-turbinate fluid/swab/wash/aspirate, pharyngeal fluid/swab/wash/aspirate, nasopharyngeal fluid/swab/wash/aspirate, oropharyngeal fluid/swab/wash/aspirate, tissue biopsy e.g. lung biopsy, cerebrospinal fluid, urine, faeces, stool, anal swab, semen, sweat, tears, processed fractions thereof and the like.

In some embodiments, the method comprises obtaining a sample. In some embodiments, the method comprises processing a sample prior to its provision to the cellulose substrate. Methods of processing different types of samples known in the art may be employed prior to their provision to the substrate. For example, methods of processing a biological sample may involve centrifugation to separate a sample into different fractions, cell lysis, extraction of DNA and/or RNA, disintegration and/or dissolving of sample, purification and/or sample concentration. In one embodiment, the method further comprises substantially dissolving/solubilizing the sample in a solvent to obtain a liquid/fluid sample. In one embodiment, the sample is provided in the form of an aqueous solution.

In some embodiments, where the analyte comprises a polynucleotide or a nucleic acid, preparing the sample comprises subjecting the sample to an amplification condition suitable for amplifying the nucleic acid analyte to a detectable level. In some embodiments, the analyte comprises a polynucleotide/nucleic acid, and the method further comprises subjecting the sample to an amplification reaction configured to amplify the polynucleotide/nucleic acid prior to the incubating step. Amplification reactions known in the art may be employed. The amplification reactions may include but are not limited to polymerase chain reaction (PCR), ligase chain reaction (LCR), loop mediated isothermal amplification (LAMP), nucleic acid sequence based amplification (NASBA), self-sustained sequence replication (3SR), rolling circle amplification (RCA) or any other process whereby one or more copies of a particular polynucleotide sequence or nucleic acid sequence may be generated from a polynucleotide template sequence or nucleic acid template sequence.

The reporter agent may be any detectable agent that is capable of indicating/reflecting a presence/absence and/or an amount of analyte that is captured by the capture agent. For example, the reporter agent may bind to an analyte and the detection of a signal effected by the reporter agent on a cellulose substrate may indicate the presence of analyte that is captured by the capture agent on the cellulose substrate. For example, the reporter agent may also compete with an analyte for binding to a capture agent and detection of signal effected by the reporter agent on a cellulose substrate may indicate that little or no analyte is captured by the capture agent on the cellulose substrate. In various embodiments therefore, the reporter agent comprises an analyte binder or a competing binder.

In one embodiment, the reporter agent comprises an analyte binder or a binding protein that is capable of binding to the analyte. In various embodiments, the reporter agent/analyte binder is capable of recognising and/or binding to the analyte. In various embodiments, the reporter agent/analyte binder has binding affinity for the analyte. In various embodiments, the reporter agent/analyte binder is specific to the analyte. In various embodiments, the affinity between the reporter agent/analyte binder and the analyte is not more than about 10⁻⁶ M, not more than about 10⁻⁷ M, not more than about 10⁻⁸ M, not more than about 10⁻⁹ M, not more than about 10⁻¹⁰, M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁶ M. In some embodiments, the affinity between the reporter agent/analyte binder and the analyte is in the micromolar range, optionally in the low micromolar range. In some embodiments, the affinity between the reporter agent/analyte binder and the analyte is in the nanomolar range, optionally in the low nanomolar range. In some embodiments, the affinity between the reporter agent/analyte binder and the analyte is in the picomolar range, optionally in the low picomolar range. In some embodiments, the affinity between the reporter agent/analyte binder and the analyte is in the femtomolar range, optionally in the low femtomolar range.

In one embodiment, the reporter agent comprises a competing binder or a binding protein that is capable of binding to the capture agent. In various embodiments, the reporter agent/competing binder is capable of recognising and/or binding to the capture agent. In various embodiments, the reporter agent/competing binder has binding affinity for the capture agent. In various embodiments, the reporter agent/competing binder is specific to the capture agent. In various embodiments, the affinity between the reporter agent/competing binder and the capture agent is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴M, not more than about 10⁻¹⁵ M or less than about 10⁻¹⁵ M. In some embodiments, the affinity between the reporter agent/competing binder and the capture agent is in the micromolar range, optionally in the low micromolar range. In some embodiments, the affinity between the reporter agent/competing binder and the capture agent is in the nanomolar range, optionally in the low nanomolar range. In some embodiments, the affinity between the reporter agent/competing binder and the capture agent is in the picomolar range, optionally in the low picomolar range. In some embodiments, the affinity between the reporter agent/competing binder and the capture agent is in the femtomolar range, optionally in the low femtomolar range.

The competing binder may be the same or different from the analyte. In one example, where the capture agent comprises CBD fused to SARS-CoV-2 Receptor Binding Domain (RBD), the competing binder comprises Angiotensin Converting Enzyme-2 (ACE2) protein while the analyte comprises neutralizing antibody that blocks the complex formation of RBD-CBD and ACE2.

In various embodiments, determining a presence or absence of said analyte captured on the one or more test zones by said capture agent comprises detecting a signal effected by a reporter agent on the one or more test zones, wherein said reporter agent: (i) comprises an analyte binder and said reporter agent is contacted with the sample to allow said reporter agent to bind to said analyte, if present, in said sample; or (ii) comprises a competing binder having affinity for said capture agent and said reporter agent is contacted with said capture agent to allow said reporter agent to bind to said capture agent. In various embodiments, therefore, the method comprises contacting a reporter agent with the sample and/or the capture agent. The contacting step may be carried out prior to or after the step of applying the sample to the one or more test zones. The contacting step may also be carried out simultaneously with or separately from the step of incubating the sample with the capture agent. For example, the reporter agent, the capture agent and the sample may be incubated together. For example, the reporter agent may be incubated with the capture agent or the sample separately from the incubation of the sample with the capture agent before dispensing both incubated contents to the one or more test zones. For example, the reporter agent may be dispensed on the one or more test zones following the immobilization of the capture agents on the test zones and/or following the application of the sample on the test zones.

In various embodiments, the reporter agent is detectable. In some embodiments, the reporter agent is detectable via a detectable analyte binder, competing binder or binding protein comprised therein. For example, an analyte binder, competing binder or binding protein may be directly detectable, or it may be indirectly detectable e.g., it is capable of e.g., interacting with a reactant to produce a detectable signal or forming a complex that generates a detectable signal. In some embodiments, the reporter agent is detectable via a label.

A label may be any molecule or moiety that facilitates detection of the reporter agent (and therefore detection of any analyte or competing binder bound to the reporter agent). A label may be capable of effecting a detectable signal, including signals that are visible and/or signals that are detectable using suitable sensor or instrumentation. Suitable labels depend on the particular assay format and/or detection system and may include those that are well known to those skilled in the art. Many labels are commercially available and may be used in embodiments of the method. Common types of labels include optical labels (e.g., fluorescent, luminescent and/or light-scattering labels, colorimetric labels, coloured molecules such as colloidal nanoparticles, molecules that generate colours during a reaction), radioactive labels, magnetic and/or electrical labels, enzymes, ligands with specific bonds, microscopic vesicles detectable by acoustic resonance, and the like. Examples of a label include, but are not limited to, a dye, a fluorescent dye, a fluorophore, a luminophore, a chromophore, a chromatogen, a chemiluminescent compound, a catalyst, an enzyme (e.g. a peroxidase), an enzymatic reagent/substrate, a biotin, a radioisotope, a mass label, a charge label, a spin label, a receptor, a ligand, a nanoparticle (e.g., gold, silver, carbon nanotubes), a colloidal metallic particle, a colloidal non-metallic particle or other moiety known in the art that can be measured by an analytical method and combinations thereof. A label can be directly detectable by the eye and/or by suitable sensor/instrumentation, or it can be detectable as a result of subsequent processes. For example, a label in the form of a dye may be directly detectable by the eye and/or by suitable sensor/instrumentation, while a label in the form of an enzyme such as horseradish peroxidase (HRP) become detectable when it produces a colour change (that may correlate with the analyte level) upon reaction with a substrate such as 3,3′,5,5″-tetramethylbenzidine (TMB).

In various embodiments therefore, detecting a signal effected by the reporter agent may be direct (e.g., by the eye and/or by suitable sensor/instrumentation) or indirect (e.g., through enzymatic reaction of a substrate by the reporter agent).

In one embodiment, the label comprises a fluorescent dye. In one embodiment, the fluorescent dye comprises an Alexa Fluor (produced by Invitrogen Corporation) based dye molecule. In one example, the fluorescent dye comprises Alexa Fluor 594. In one embodiment, the label comprises an enzyme that is capable of producing a colorimetric signal/readout. In one embodiment, the enzyme comprises a peroxidase. In one embodiment, the peroxidase comprises HRP. Advantageously, the use of HRP is convenient as it can produce a colorimetric signal/readout that is visible/discernible by the naked/unaided eye. The optical density of the colorimetric signal/readout can further be quantified/measured spectrophotometrically. Furthermore, HRP is capable of amplifying a signal and increasing the detectability of a reporter agent. HRP is also stable, and relatively resistant to heat and organic solvent.

A label may be coupled/attached directly to an analyte binder, competing binder or binding protein in a reporter agent, or it may be coupled/attached indirectly to an analyte binder, competing binder or binding protein in a reporter agent through one or more bridges or secondary reporter agents. For example, a label may be coupled/conjugated directly to an analyte binder, competing binder or binding protein in a reporter agent. In one embodiment, a label in the form of a fluorescent dye is coupled/conjugated to a competing binder to form the reporter agent. For example, a label may be coupled to an analyte binder, competing binder or binding protein through a biotin-streptavidin bridge or biotin-avidin bridge, or through a secondary antibody. In one embodiment, an analyte binder or binding protein is tagged with biotin and allowed to interact with HRP-conjugated streptavidin (SA-HRP) to produce a reporter agent in the form of a HRP-labelled analyte binder or binding protein coupled through a biotin-streptavidin bridge. Advantageously, an indirect coupling/attachment of a label to an analyte binder, competing binder or binding protein through e.g., the use of one or more bridges or secondary reporter agents may enhance/amplify a signal and thereby improve the detection sensitivity and/or the limit of detection of the method. For example, biotin and streptavidin has a dissociation constant (K_{d}) in the femtomolar range and the strong affinity between the two molecules allow more analytes to be coupled successfully to a label for detection. In one embodiment therefore, the reporter agent comprises a label coupled to an analyte binder, competing binder or binding protein through a biotin-streptavidin or biotin-avidin bridge. Advantageously, the biotin-streptavidin and biotin-avidin systems not only enhance/amplify a signal, but the systems are also versatile and compatible with a wide variety of analyte binders or binding proteins and labels.

In various embodiments, the reporter agent comprises an analyte binder, competing binder or binding protein and a label coupled thereto. In various embodiments, the reporter agent may further comprise one or more bridges. In various embodiments, the reporter agent comprises an analyte binder, competing binder or binding protein coupled to a label through one or more bridges. In various embodiments, the analyte binder, competing binder or binding protein is tagged with a biotin or biotinylated. The analyte binder, competing binder or binding protein may be further coupled or fused to one or more partners or moieties, e.g., a maltose-binding protein (MBP) to increase biotin accessibility. In various embodiments, the label is coupled/conjugated to streptavidin or avidin. In various embodiments, the reporter agent comprises an analyte binder, competing binder or binding protein coupled to a label through a biotin-streptavidin or biotin-avidin bridge. It may be appreciated that a label may already be present in the reporter agent e.g., at the incubating/contacting step or the reporter agent may be devoid of a label and is coupled to a label e.g., during or after the incubating/contacting step. In some embodiments therefore, the method comprises providing a reporter agent comprising a label. In some embodiments, the method comprises a step of adding/coupling a label (e.g., a streptavidin-conjugated label or a streptavidin-conjugated label) to an analyte binder, competing binder or binding protein (e.g., a biotinylated analyte binder, competing binder or binding protein), and optionally incubating/contacting the two, to obtain a reporter agent comprising/coupled to a label or a labelled reporter agent. The label adding/coupling step may be performed before, during or after the sample incubating step. The label adding/coupling step may be performed prior to the step of incubating the reporter agent with the capture agent. In some embodiments, a label is incubated with the sample and the analyte binder, competing binder or binding protein to obtain a reporter agent comprising/coupled a label or a labelled reporter agent (e.g., through biotin-streptavidin or biotin-avidin mediated coupling of the label to the analyte binder, competing binder or binding protein).

In various embodiments, the method further comprises adding a reagent capable of producing a detectable signal upon interaction/reaction with the reporter agent after the applying step. Suitable reagents depend on the particular reporter agent (e.g., the particular analyte binder, competing binder or binding protein and/or the particular label). For example, where the reporter agent comprises a HRP label, the reagent may be a HRP substrate that gives a detectable (e.g., coloured) end product upon reaction with HRP. In some embodiments therefore, where the label comprises HRP, the method comprises adding a reagent selected from the group consisting of o-Phenylene Diamine (OPD), 2,2′-azino-bis(3-ethylbenzthiazoline-6-sulphonate (ABTS), 3-amino-9-ethylcarbazole (AEC), 4-Chloro-1-Naphtol (4-CN), 3,3″,5,5″-tetramethylbenzidine (TMB), 3,3′-DiAminoBenzimidine (DAB), 10-Acetyl-3,7-Dihydroxyphenoxazine (ADHP), Resazurin, Luminol, Uptilight tyramide and combinations thereof.

In various embodiments, the method comprises a step of adding a label (e.g., SA-HRP) prior or after the applying step and a subsequent step of adding a reagent (e.g., TMB) for expressing the signal.

In various embodiments, the signal/readout effected or produced by the reporter agent or label include an optical signal/readout (e.g. colorimetric signal/readout, fluorescence, luminescence such as chemiluminescence, electrochemiluminescence and photoluminescence etc.), radioactivity, infrared radiation, resonance energy transfer (FRET), emission signal/readout, magnetic and/or electrical signal/readout, acoustic signal/readout and any other forms of signal/readout that can be produced by a label or an analyte binder, competing binder or binding protein as described herein. In some embodiments, the signal/readout comprises a colorimetric signal/readout. In some embodiments, the colorimetric signal/readout is visible/discernible/distinguishable to the naked/unaided eye. In some embodiments, the signal/readout comprises a fluorometric signal/readout. In some embodiments, the intensity of the fluorometric/colorimetric signal/readout correlates with the level/amount/concentration of the analyte in the sample. In some embodiments, the intensity of the fluorometric/colorimetric signal/readout positively correlates with the level/amount/concentration of the analyte in the sample i.e., the higher the intensity of the fluorometric/colorimetric signal/readout, the higher the level/amount/concentration of the analyte in the sample. For example, a darker or more intense colour, or a higher fluorescence intensity produced by a reporter agent comprising an analyte binder may indicate that a sample contains a higher level/amount/concentration of the analyte than a lighter/fainter or less intense colour or a lower fluorescence intensity. In some embodiments, the intensity of the of the fluorometric/colorimetric signal/readout negatively correlates with the level/amount/concentration of the analyte in the sample (e.g., in a competitive binding assay) i.e., the higher the intensity of the fluorometric/colorimetric signal/readout, the lower the level/amount/concentration of the analyte in the sample. For example, a darker or more intense colour, or a higher fluorescence intensity produced by a reporter agent comprising a competing binder (e.g., in a competitive binding assay) may indicate that a sample contains a lower level/amount/concentration of the analyte than a lighter/fainter or less intense colour or a lower fluorescence intensity.

In some embodiments, the signal/readout is quantifiable. In some embodiments, detecting a signal/readout effected or produced by the reporter agent or label comprises capturing the signal/readout with a camera and optionally analysing the captured image to e.g., measure/quantify the intensity of the signal/readout. The image analysis may be carried out, for example, by use of ImageJ software or other suitable imaging software programs or algorithms.

In various embodiments, where the reporter agent comprises an analyte binder, the sample is incubated with the reporter agent under conditions that allow the analyte, if present in the sample, to be bound by the reporter agent. In various embodiments, the sample is incubated with the reporter agent for a sufficient time to allow binding of any analyte therein to the reporter agent. The duration of incubation/interaction may depend on the binding affinity of the reporter agent for the analyte as well as the concentration of the analyte. In various embodiments, the sample is incubated with the reporter agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds, not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the sample is incubated with the reporter agent for about 15 seconds to about 300 seconds, about 15 seconds to about 180 seconds, from about 30 seconds to about 180 seconds, from about 45 seconds to about 180 seconds, from about 60 seconds to about 180 seconds, from about 15 seconds to about 120 seconds, from about 30 seconds to about 120 seconds, from about 45 seconds to about 120 seconds or from about 60 seconds to about 120 seconds. In one embodiment, the sample is incubated with the reporter agent for more than about 10 seconds. In one embodiment, the sample is incubated with the reporter agent for at least about 60 seconds. Advantageously, in various embodiments, the sample is allowed a sufficient amount of time to interact with the reporter agent for the effective formation of a reporter agent-analyte complex. More analytes may be successfully bound to a reporter agent at the early stage and this may improve the detectability of the analytes in the later detection stage of the method. Consequently, sensitivity of the assay/method may be increased and false negative results may be reduced. The limit of detection may also be improved.

In various embodiments, where the reporter agent comprises a competing binder, the reporter agent is incubated with the capture agent under conditions that allow the reporter agent to be bound by the capture agent. The duration of interaction/incubation may depend on the dissociation constant (Kd) between the reporter agent and the capture agent, and the Kd between the analyte and the capture agent if the sample is incubated together with the reporter agent and the capture agent. In various embodiments, the capture agent is incubated with the reporter agent for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds or not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the capture agent is incubated with the reporter agent for about 15 seconds to about 300 seconds, about 15 seconds to about 180 seconds, about 30 seconds to about 180 seconds, about 45 seconds to about 180 seconds, about 60 seconds to about 180 seconds, about 15 seconds to about 120 seconds, about 30 seconds to about 120 seconds, about 45 seconds to about 120 seconds or about 60 seconds to about 120 seconds. In one embodiment, the capture agent is incubated with the reporter agent for more than about 10 seconds. In one embodiment, the capture agent is incubated with the reporter agent for at least about 60 seconds. Advantageously, in various embodiments, the reporter agent is allowed a sufficient amount of time to interact with the capture agent for the effective formation of a reporter agent-capture agent complex. More reporter agents can be successfully bound to a capture agent to be captured onto the cellulose substrate. False positive results may be reduced.

In various embodiments, the incubating step is carried out in solution. In various embodiments, the incubating step is carried out in a solvent. In various embodiments, the incubating step comprises adding the sample, the reporter agent and/or the capture agent to a solvent. In various embodiments, the solvent offers solubility to the analyte, the reporter agent and/or the capture agent. In various embodiments, the solvent is capable of solubilizing the analyte, the reporter agent and/or the capture agent. Examples of suitable solvents include, but are not limited to, phosphate-buffered saline (PBS); lysis buffer (e.g., buffer containing surfactant or detergent); buffer containing stabilizer e.g., glycerol, sucrose, trehalose; biological fluids (e.g., blood, plasma, saliva, etc.); water; liquid component of food; liquid component of environmental samples; and the like etc. In various embodiments, the solvent is selected from the group consisting of: buffer solution, biological fluid, water, liquid component of food, liquid component of environmental samples and combinations thereof.

After incubation, the incubated sample may be applied to/contacted with a cellulose substrate to allow any reporter agent-analyte complex to be captured onto the cellulose substrate by the capture agent or to allow any reporter agent-capture agent complex to be captured onto the cellulose substrate.

In various embodiments, the capture agent is capable of recognising and/or binding to the analyte. In various embodiments, the capture agent has binding affinity for the analyte. In various embodiments, the capture agent is specific to the analyte. In various embodiments, the capture agent comprises an analyte binder or binding protein that is capable of binding to the analyte. In various embodiments, the affinity between the capture agent and the analyte is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the capture agent and the analyte is in the micromolar range, optionally in the low micromolar range. In some embodiments, the affinity between the capture agent and the analyte is in the nanomolar range, optionally in the low nanomolar range. In some embodiments, the affinity between the capture agent and the analyte is in the picomolar range, optionally in the low picomolar range. In some embodiments, the affinity between the capture agent and the analyte is in the femtomolar range, optionally in the low femtomolar range.

In various embodiments, the capture agent is capable of recognising and/or binding to the reporter agent e.g., a reporter agent comprising a competing binder having affinity for the capture agent. In various embodiments, the capture agent has binding affinity for the reporter agent. In various embodiments, the capture agent is specific to the reporter agent. In various embodiments, the affinity between the capture agent and the reporter agent is not more than about 10⁻⁶ M, not more than about 10⁻⁷M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the capture agent and the reporter agent is in the micromolar range, optionally in the low micromolar range. In some embodiments, the affinity between the capture agent and the reporter agent is in the nanomolar range, optionally in the low nanomolar range. In some embodiments, the affinity between the capture agent and the reporter agent is in the picomolar range, optionally in the low picomolar range. In some embodiments, the affinity between the capture agent and the reporter agent is in the femtomolar range, optionally in the low femtomolar range.

In various embodiments, the capture agent comprises a cellulose binding domain (CBD). CBD is a protein domain that is present in many carbohydrate active enzymes. CBDs have been classified into at least 13 families named I-XIII according to their amino acid sequence similarities, with most of the reported CBDs belonging to families I, II, and III. The CBD that may be incorporated in the capture agent is not particularly limited to CBDs of certain families. In various embodiments, the CBD may be selected from the group consisting of: a family I CBD, a family II CBD, a family III CBD, a family IV CBD, a family V CBD, a family VI CBD, a family VII CBD, a family VIII CBD, a family IX CBD, a family X CBD, a family XI CBD, a family XII CBD, a family XIII CBD and combinations thereof. In one embodiment, the CBD comprises a family III CBD. In one embodiment, the CBD comprises a CBD from a CBD3a family. The CBD may be a naturally occurring CBD, or it may be a modified/synthesised CBD having affinity for cellulose.

In various embodiments, the CBD comprises from about 20 to about 200 amino acid residues, from about 30 to about 40 amino acid residues, from about 80 to about 110 amino acid residues, from about 90 to about 100 amino acid residues, from about 120 to about 180 amino acid residues or from about 130 to about 180 amino acid residues. In some embodiments, the CBD comprises from about 150 to about 170 amino acid residues. In one embodiment, the CBD comprises about 160 amino acid residues.

In some embodiments, the CBD comprises/consists of the SEQ ID NO: 1 (PVSGNLKVEFYNSNPSDTTNSINPQFKVTNTGSSAIDLSKLTLRYYYTVDGQK DQTFWCDHAAIIGSNGSYNGITSNVKGTFVKMSSSTNNADTYLEISFTGGTLE PGAHVQIQGRFAKNDWSNYTQSNDYSFKSASQFVEWDQVTAYLNGVLVWG KEP) or portions, optionally linear portions, thereof or a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto. In some embodiments, the CBD is encoded by SEQ ID NO: 2 (CCGGTATCAGGCAATTTGAAGGTTGAATTCTA CAACAGCAATCCTTCAGATACTACTAACTCAATCAATCCTCAGTTCAAGGTT ACTAATACCGGAAGCAGTGCAATTGATTTGTCCAAACTCACATTGAGATATT ATTATACAGTAGACGGACAGAAAGATCAGACCTTCTGGTGTGACCATGCTG CAATAATCGGCAGTAACGGCAGCTACAACGGAATTACTTCAAATGTAAAAG GAACATTTGTAAAAATGAGTTCCTCAACAAATAACGCAGACACCTACCTTG AAATAAGCTTTACAGGCGGAACTCTTGAACCGGGTGCACATGTTCAGATAC AAGGTAGATTTGCAAAGAATGACTGGAGTAACTATACACAGTCAAATGACT ACTCATTCAAGTCTGCTTCACAGTTTGTTGAATGGGATCAGGTAACAGCAT ACTTGAACGGTGTTC TTGTATGGGGTAAAGAACCC) or portions, optionally linear portions, thereof or a sequence sharing at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity thereto. A portion, optionally a linear portion, of SEQ ID NO:1 or 2 may comprise one or more stretches of the amino acid residues in SEQ ID NO: 1 or one or more stretches of the bases or nucleotides in SEQ ID NO: 2 that is capable of encoding a protein/peptide product that can bind to cellulose with high affinity. The stretch of residues or bases/nucleotides may comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145 or at least about 150 successive residues or bases/nucleotides in SEQ ID NO:1 or 2.

In various embodiments, the CBD has a binding affinity for cellulose that is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the CBD has a binding affinity for cellulose that is about 1 μM or less. In some embodiments, the CBD has a binding affinity for cellulose that is from about 0.5 μM to about 1 μM. In some embodiments, the CBD comprises a family I CBD having a binding affinity for cellulose that is from about 0.5 μM to about 1 μM.

In some embodiments, the CBD is located at an end or at an extremity of the capture agent. In some embodiments, the CBD is attached or coupled to an end or an extremity of an analyte binder or a binding protein in the capture agent. In some embodiments, the CBD is attached or coupled to a terminus of the analyte binder or binding protein for the analyte. In some embodiments, the CBD is attached or coupled to a C-terminus of the analyte binder or binding protein for the analyte. In various embodiments, the positioning of the CBD within the capture agent advantageously allows the capture agent to be immobilised onto the cellulose substrate in an optimal orientation, e.g., with the binding faces facing away from the cellulose substrate and/or towards the applied sample, such that the binding faces are available for contacting and binding to the analyte. Consequently, more capture agents can bind the analytes successfully, and detectability of the analytes may be increased. The sensitivity of the assay/method may be increased and false negative results can be reduced. The limit of detection may also be improved. In some embodiments therefore, a plurality of capture agents is immobilised on the cellulose substrate in a substantially uniform orientation.

The capture agent may be incubated with the sample prior to the applying step and/or immobilised on the cellulose substrate prior to the applying step.

In some embodiments, the capture agent is incubated with the sample prior to the applying step. The capture agent may be incubated with the sample separately from the reporter agent (e.g., before and after the sample is incubated with the reporter agent), and/or it may be incubated with the sample together with the reporter agent.

In some embodiments, where the reporter agent comprises an analyte binder, the sample is first incubated/contacted with the capture agent to form a capture agent-analyte complex and then subsequently incubated/contacted with the reporter agent to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate (via affinity between the CBD of the capture agent and the cellulose substrate) for detection. In some embodiments, where the reporter agent comprises an analyte binder, the sample is first incubated/contacted with the reporter agent to form a reporter agent-analyte complex and then subsequently incubated/contacted with the capture agent to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate for detection. In some embodiments, where the reporter agent comprises an analyte binder, the sample is incubated/contacted with both the reporter agent and the capture agent, and optionally a label, at the same time (i.e., together) to form a full sandwich complex in the form of a reporter agent-analyte-capture agent complex. The sample containing the full sandwich complex may then be applied/dispensed onto the cellulose substrate to allow the full sandwich complex to be immobilised on the cellulose substrate for detection. In some embodiments therefore, wherein the capture agent is incubated with the sample prior to the applying step, the analyte that is bound to the reporter agent is part of a reporter agent-analyte-capture agent complex (i.e., a full sandwich complex) prior to the applying step.

In some embodiments therefore, where said reporter agent comprises an analyte binder, the method comprises incubating the reporter agent and the capture agent with said sample prior to the applying step and said analyte when present, is bound to the reporter agent to form part of a reporter agent-analyte-capture agent complex prior to the applying step.

In some embodiments, where the reporter agent comprises a competing binder, the sample is first incubated/contacted with the capture agent to form a capture agent-analyte complex and then subsequently incubated/contacted with the reporter agent to form a capture agent-reporter agent complex. The sample containing both the capture agent-analyte complex and the capture agent-reporter agent complex may then be applied/dispensed onto the cellulose substrate to allow the complexes to be immobilised on the cellulose substrate (via affinity between the CBD of the capture agent and the cellulose substrate) for detection. In some embodiments, where the reporter agent comprises a competing binder, the reporter agent is first incubated/contacted with the capture to form a capture agent-reporter agent complex and then subsequently incubated/contacted with the sample to form a capture agent-analyte complex. The sample containing both the capture agent-analyte complex and the capture agent-reporter agent complex may then be applied/dispensed onto the cellulose substrate to allow the complexes to be immobilised on the cellulose substrate for detection. In some embodiments, where the reporter agent comprises a competing binder, the sample is incubated/contacted with both the reporter agent and the capture agent, and optionally a label, at the same time (i.e., together) to form both the capture agent-analyte complex and the capture agent-reporter agent complex. The sample containing both the capture agent-analyte complex and the capture agent-reporter agent complex may then be applied/dispensed onto the cellulose substrate to allow the complexes to be immobilised on the cellulose substrate for detection.

In some embodiments therefore, where said reporter agent comprises a competing binder, the method comprises incubating said capture agent with said reporter agent and/or said sample prior to the applying step to form a reporter agent-capture agent complex and/or an analyte-capture agent complex prior to the applying step

In various embodiments, the sample is incubated with the capture agent under conditions that allow the analyte, if present in the sample, to be bound by the capture agent. In various embodiments, the capture agent is incubated with the sample for not less than about 15 seconds, not less than about 30 seconds, not less than about 45 seconds, not less than about 60 seconds, not less than about 75 seconds, not less than about 90 seconds, not less than about 105 seconds, not less than about 120 seconds, not less than about 135 seconds, not less than about 150 seconds, not less than about 165 seconds or not less than about 180 seconds, not less than about 195 seconds, not less than about 210 seconds, not less than about 225 seconds, not less than about 240 seconds, not less than about 255 seconds, not less than about 270 seconds, not less than about 285 seconds or not less than about 300 seconds. In various embodiments, the capture agent is incubated with the sample for about 15 seconds to about 300 seconds, about 15 seconds to about 180 seconds, about 30 seconds to about 180 seconds, about 45 seconds to about 180 seconds, about 60 seconds to about 180 seconds, about 15 seconds to about 120 seconds, about 30 seconds to about 120 seconds, about 45 seconds to about 120 seconds or about 60 seconds to about 120 seconds. In one embodiment, the capture agent is incubated with the sample for more than about 10 seconds. In one embodiment, the capture agent is incubated with the sample for at least about 60 seconds. Advantageously, in various embodiments, the sample is allowed a sufficient amount of time to interact with the capture agent for the effective formation of a capture agent-analyte complex or if the reporter agent is present, a reporter agent-analyte-capture agent complex. More analytes can be successfully bound to a capture agent to be captured onto the cellulose substrate. This may improve the detectability of the analytes. Consequently, sensitivity of the assay/method may be increased and false negative results may be reduced. The limit of detection may also be improved.

In some embodiments, the capture agent is immobilised on the cellulose substrate prior to the applying step. In some embodiments, the method comprises adding/dispensing capture agents onto the cellulose substrate to allow the capture agents to be immobilised on the cellulose substrate before the applying step e.g., before, after or at the same time with the incubating step. The capture agent may be pre-immobilised on the cellulose substrate and/or it may be added/dispensed onto the cellulose substrate prior to the applying step. In some embodiments, the method comprises providing a cellulose substrate with the capture agents immobilised/disposed thereon e.g., before the incubating step or applying step. Advantageously, embodiments of the method are suitable for use with embodiments of the apparatus described herein comprising a cellulose substrate, which allows for the application to the substrate of a full-sandwich complex in the form of a reporter agent-analyte-capture agent complex, a half-sandwich complex in the form of a reporter agent-analyte complex, or in the case of a competitive binding assay, a capture agent-reporter agent complex and/or a capture agent-analyte complex.

In various embodiments, where said reporter agent comprises an analyte binder, the method comprises incubating the reporter agent with said sample and immobilizing said capture agent on the one or more test zones of said cellulose substrate prior to the applying step and said analyte when present, is bound to the reporter agent to form a reporter agent-analyte complex prior to the applying step.

In various embodiments, where said reporter agent comprises a competing binder, the method comprises immobilizing said capture agent on the one or more test zones of said cellulose substrate prior to the applying step and dispensing the reporter agent on the one or more test zones after the applying step.

Embodiments of the method employing a capture agent having CBD have several advantages. CBD has very high affinity towards cellulose substrate in which the interaction or binding between the CBD and the cellulose can happen in less than a second. This increases the efficiency of the method. For example, after a full sandwich complex (i.e., a reporter agent-analyte-capture agent complex) is applied to the cellulose substrate, less waiting time may be required before detection of sandwich complex captured by the cellulose substrate can be made. For example, where a capture agent is applied to the cellulose substrate, less waiting time may also be required to immobilise the capture agent onto the cellulose substrate before the next step, e.g., the application of the sample to the cellulose substrate, may be carried out. Further, the immobilisation of a conventional capture agent devoid of a CBD onto a cellulose substrate typically occurs spontaneously through hydrophobic and electrostatic interactions and such interactions offer only moderate strength. Thus, some capture agents may be lost. There is also no control over the orientation of the capture agents on the cellulose substrate. As a result, the capture agents are immobilised on the substrate in random orientations with the binding faces facing non-uniform directions. Collectively, the loss of capture agents and random orientations of the capture agents on the substrate may reduce the amount of capture agents available for binding to the analytes or reporter agents and assay sensitivity and/or accuracy may be reduced. By contrast, embodiments of the capture agent comprising a CBD has much higher affinity for the cellulose substrate, thus allowing more capture agents to be immobilised on the cellulose substrate. Thus, in various embodiments, less capture agents are lost, if any. The specific binding of the CBD to the cellulose may also advantageously orient the capture agents such that their binding faces are directed towards the applied analytes or reporter agents. Collectively, this can increase the availability of the capture agents for capturing analytes or reporter agents. The sensitivity of a non-competitive assay/method may be increased and false negative results may be reduced. The limit of detection may also be improved. In a competitive assay/method, the accuracy of the method may also be increased and false positive results may be reduced. Further, with less capture agents being lost because of inadequate binding strength, the use of the capture agents may also be better optimised. Less capture agents may be required to capture a given amount of analyte or reporter agent, reducing wastage of resources and cost of the method.

Embodiments of the method employing a capture agent having CBD is also a relatively safe and easy alternative to immobilize proteins on a cellulose substrate as compared to using functionalized cellulose paper for immobilizing proteins. The synthesis of functionalized cellulose paper requires periodate, which is a dangerous chemical, for oxidation of cellulose to make carboxylic groups.

In various embodiments, the amount of capture agents used is not more than about 1000-fold molar excess, not more than about 900-fold molar excess, not more than about 800-fold molar excess, not more than about 700-fold molar excess, not more than about 600-fold molar excess, not more than about 500-fold molar excess, not more than about 400-fold molar excess, not more than about 300-fold molar excess, not more than about 200-fold molar excess, not more than about 100-fold molar excess, not more than about 90-fold molar excess, not more than about 80-fold molar excess, not more than about 70-fold molar excess, not more than about 60-fold molar excess, not more than about 50-fold molar excess, not more than about 40-fold molar excess, not more than about 30-fold molar excess, not more than about 20-fold molar excess, not more than about 10-fold molar excess, not more than about 9-fold molar excess, not more than about 8-fold molar excess, not more than about 7-fold molar excess, not more than about 6-fold molar excess or not more than about 5-fold molar excess of the analytes or of the typical amount range of analytes that is contained in a typical sample of a similar/identical type as the sample. In some embodiments, the capture agent is not more than 1000-fold molar excess, optionally not more than 100-fold molar excess, further optionally not more than 60-fold molar excess of said analyte. In various embodiments, the ratio of the moles of the capture agents to the moles of the analyte (or the typical molar range of analyte that is contained in a typical sample of a similar/identical type as the sample) is not more than about 1000, not more than about 900, not more than about 800, not more than about 700, not more than about 600, not more than about 500, not more than about 400, not more than about 300, not more than about 200, not more than about 100, not more than about 90, not more than about 80, not more than about 70, not more than about 60, not more than about 50, not more than about 40, not more than about 30, not more than about 20, not more than about 10, not more than about 9, not more than about 8, not more than about 7, not more than about 6 or not more than about 5. Surprisingly, embodiments of the method are demonstrated to have better sensitivity than comparative examples using a greater excess of the capture agents. The surprising results may be attributed to the longer incubation time between the sample and the reporter agents and/or the capture agents in embodiments of the method. As compared to the use of an abundant excess of capture agents, a longer incubation time may be more effective in maximising/increasing the amount of full sandwich complex captured successfully on the cellulose substrate, and therefore more effective n increasing assay sensitivity. Resource wastage and cost are also reduced in embodiments of the method.

In various embodiments, the capture agent and/or the reporter agent may comprise an analyte binder or a binding protein for the analyte. The analyte binder or the binding protein may be capable of binding to the analyte. In various embodiments, the analyte binder or the binding protein is capable of binding to the analyte with substantially high specificity and/or high affinity. In various embodiments, the affinity between the analyte binder or the binding protein is not more than about 10⁻⁶ M, not more than about 10⁻⁷ M, not more than about 10⁻⁸ M, not more than about 10⁻⁹ M, not more than about 10⁻¹⁰ M, not more than about 10⁻¹¹ M, not more than about 10⁻¹² M, not more than about 10⁻¹³ M, not more than about 10⁻¹⁴ M, not more than about 10⁻¹⁵ M or less than about 10⁻¹⁵ M. In some embodiments, the affinity between the analyte binder or the binding protein is in the micromolar range, optionally in the low micromolar range. In some embodiments, the affinity between the analyte binder or the binding protein is in the nanomolar range, optionally in the low nanomolar range. In some embodiments, the affinity between the analyte binder or the binding protein is in the picomolar range, optionally in the low picomolar range. In some embodiments, the affinity between the analyte binder or the binding protein is in the femtomolar range, optionally in the low femtomolar range. In some embodiments, the reporter agent and/or capture agent comprises an analyte binder or a binding protein having picomolar affinity for the analyte.

The analyte binder or the binding protein may be an antibody or a non-antibody. Examples of analyte binders or binding proteins include, but are not limited to antibodies (polyclonal and/or monoclonal), antigen binding proteins, peptides, aptamers, haptens, receptors, engineered proteins, engineered protein scaffolds such as AdNectin, Affibody, Anticalin, Knottin, DARPin, Kunitz, affitins, fibronectins and other organic and/or polymeric scaffolds, fragments thereof (e.g., antigen- or analyte-binding fragments thereof) and the like. As used herein, the term “antibody” refers to an immunoglobulin or fragment thereof, and encompasses any polypeptide comprising an antigen-binding fragment or an antigen-binding domain. The term includes but is not limited to polyclonal, monoclonal, monospecific, polyspecific (such as bi-specific), humanized, human, single-chain, chimeric, synthetic, recombinant, hybrid, mutated, grafted, and in vitro generated antibodies. The term “antibody” may include antibody fragments such as Fab, F(ab′)2, Fv, scFv, Fd, dAb, and other antibody fragments that retain antigen-binding function. An antibody is not necessarily from any particular source, nor is it produced by any particular method.

In some embodiments, the analyte binder or the binding protein comprises a non-antibody analyte binder or a non-antibody binding protein. In some embodiments, the non-antibody analyte binder or the non-antibody binding protein comprises a protein scaffold. In some embodiments, the protein scaffold comprises a protein originating, based on or derived from Sulfolobus genera. In some embodiments, the protein scaffold comprises an affitin. Affitins are highly stable engineered affinity proteins. As background, affitins are originally derived from the 7 kDa DNA-binding polypeptides, comprising Sac7d and Sso7d, from Sulfolobus genera. Positively charged amino acids are removed from affitin creating reduced charge affitins (rcSac7d and rcSso7d) that can bind to other types of molecules other than DNA. By randomizing the amino acids on the binding surface of the polypeptides e.g., rcSac7d or rcSso7d, and subjecting the resulting protein library to rounds of yeast surface display, the affinity can be directed towards various targets including peptides, proteins, viruses, and bacteria. Advantageously, affitins are small (typically about 7 kDa), durable, highly soluble, highly selective, cost effective, resistant to extreme alkaline pH and chemically and thermally stable. In various embodiments therefore, the analyte binder or binding protein, optionally the non-antibody analyte binder or non-antibody binding protein has one or more of the following properties: small size (for example, smaller than a typical antibody having a size of 130-150 kDa), durable (e.g., able to withstand many cycles of purification), highly soluble, highly selective, cost effective, resistant to extreme alkaline pH, chemically stable and thermally stable. In various embodiments, the affitin is based on or derived from a protein of the Sul7d family. In various embodiments, the affitin is Sac7d-based, or Sac7d-derived, or Sso7d-based or Sso7d-derived. In one embodiment, the affitin is Sso7d-based or Sso7d-derived. Embodiments of the affitin may be wholly or partially isolated from a naturally occurring Sso7d or Sac7d directly, or more typically, it may be wholly synthesized using the amino acid sequence of a Sso7d or Sac7d as a prototype/reference (and then subjected to further mutation or modification and selection by yeast surface display to generate an affitin having affinity to the analyte of interest). In some embodiments, the affitin comprises rcSso7d protein.

In various examples, the affitin is specific to a coronavirus protein, optionally SARS-CoV-2 protein, further optionally a SARS-CoV-2 nucleocapsid protein. In various embodiments therefore, the analyte comprises a coronavirus protein, optionally SARS-CoV-2 protein. In one embodiment, the analyte comprises SARS-CoV-2 nucleocapsid protein or fragments thereof. In various embodiments, the method is a method of detecting coronavirus, optionally severe acute respiratory syndrome coronavirus 2 (SARS-CoV-2) in a sample or in a subject from which the sample is obtained from. In various embodiments, the method is a method of detecting an infection, optionally a virus infection, further optionally a coronavirus infection, further optionally COVID-19 infection in a sample or in a subject from which the sample is obtained from.

In various embodiments, the method comprises a diagnostic method. The method may be implemented/performed as an immunoassay method, an enzyme-linked immunosorbent assay (ELISA) method, a vertical flow assay/method, a rapid diagnostic test method, a point-of-care diagnostic method or the like. In various embodiments, the method is a lateral flow assay method or a vertical flow assay method. In one embodiment, the method comprises a point-of-care vertical flow method. In one example, the method is performed using a form of vertical flow assay whereby the sample and the reagent solutions were flowed in a top-to-bottom direction. In various embodiments, the method is implemented/performed in a point-of-care device.

Advantageously, embodiments of the method provide rapid results. In various embodiments, the results (e.g. the presence, absence, amount, relative amount of the analyte) may be obtained within about 4 hours, within about 3.5 hours, within about 3 hours, within about 2.5 hours, within about 2 hours, within about 1.5 hour, within about 1 hour, within about 55 minutes, within about 50 minutes, within about 45 minutes, within about 40 minutes, within about 35 minutes, within about 30 minutes, within about 25 minutes, within about 20 minutes, within about 15 minutes, within about 10 minutes, within about 9 minutes, within about 8 minutes, within about 7 minutes, within about 6 minutes or within about 5 minutes from the start of the incubating step. In one embodiment, the presence or absence of the analyte in the sample is determined within 15 minutes from the start of the incubating step.

The method may further comprise applying/contacting a control sample to the cellulose substrate to obtain an indication on whether the method is correctly performed. The control sample may comprise a positive control sample, a negative control sample, or both. As may be appreciated, a detection or an immobilization of a positive control sample may indicate that the method is correctly performed, for example, a binding of the reporter and capture agents to the analyte is successful e.g., during the incubation step and that the result obtained is not a false negative due to unsuccessful binding e.g., during the incubation step. No detectable immobilization of a negative control sample may indicate that the method is correctly performed, for example, there is no non-specific interaction and that the result obtained is not a false positive. In some embodiments, the method comprises processing the control sample prior to applying/contacting it with the cellulose substrate. As may be appreciated, methods of processing the sample, as herein disclosed before, may apply to the control sample.

Embodiments of the method comprising an incubation step and/or use of a capture agent comprising a CBD advantageously lead to improvement in analyte detection sensitivity. In a comparative example, the pre-incubation of analytes, reporter agents and/or CBD-tagged capture reagents comprising CBD to form complexes before subsequent application of the complexes to cellulose paper was found to result in significantly greater colorimetric intensity production as compared to the direct application of analytes and reporter agents (without incubation) to a cellulose paper with CBD-tagged capture reagents immobilised thereon. In the direct application example, the analytes, reporter agent and CBD-tagged capture reagents have only a very short interaction time (˜1-3 sec) with each other. Hence, the leads to inefficient binding of the reporter agents to the analytes and also the inefficient capturing of the analytes to the cellulose paper by the capture agents. Consequently, the sensitivity of the assay is relatively low, as evidenced by the low colorimetric intensity production. In contrast, without being bound by theory, it is believed that incubation promotes the effective binding between the analytes and reporter agents and/or capture agents by allowing for sufficient interaction time between the analytes and reporter agents and/or capture agents. This increases the amount of full sandwich complexes successfully formed and captured onto the cellulose substrate, thereby increasing the analyte detection sensitivity. In another comparative example, a capture agent without CBD (specifically rcSso7d without CBD) was found to bind to cellulose paper 2000 times less than a capture agent comprising a CBD (specifically CBD-tagged rcSso7d) at 1 to 10 seconds. This finding indicates that a capture agent comprising a CBD is able to bind to a cellulose substrate much more efficiently than a capture agent devoid of a CBD. Analyte detection sensitivity is therefore increased with the use of a capture agent comprising a CBD, since a greater amount of capture agents can be successfully bound to the cellulose substrate. Collectively, an incubation step and/or use of a capture agent comprising a CBD lead to significant improvement in analyte detection sensitivity.

### System/Kit

In various embodiments, there is provided a system for detecting an analyte in a sample, the system comprising: an incubation mixture comprising said sample and a reporter agent configured to bind to said analyte; and the apparatus disclosed herein containing a cellulose substrate configured to capture the analyte that is bound to said reporter agent, via a capture agent comprising a cellulose binding domain (CBD), wherein said capture agent is: (i) present in the incubation mixture; and/or (ii) pre-immobilised on said cellulose substrate, and wherein the presence or absence of said analyte captured on said cellulose is determinable by detection via the reporter agent. In various embodiments, there is provided a system for detecting an analyte in a sample, the system comprising: an incubation mixture comprising said sample and a reporter agent configured to bind to a capture agent comprising a cellulose binding domain (CBD); and the apparatus disclosed herein containing a cellulose substrate configured to capture the analyte and reporter agent, via said capture agent, wherein said capture agent is: (i) present in the incubation mixture; and/or (ii) pre-immobilised on said cellulose substrate, and wherein the presence or absence of said analyte captured on said cellulose is determinable by detection via the reporter agent.

In various embodiments, there is provided a kit for detecting an analyte in a sample, the kit comprising: an apparatus disclosed herein; an incubation solution/mixture/solvent (e.g., for incubating and/or binding said sample and a reporter agent that is configured to bind to said analyte in a non-competitive binding assay, or for incubating and/or binding said sample and a reporter agent that is configured to bind to said capture agent in a competitive binding assay); and a cellulose substrate (e.g., configured to capture the analyte that is bound to said reporter agent in a non-competitive assay or configured to capture both the analyte and reporter agent in a competitive binding assay). In some embodiments, the kit further comprises a capture agent. In some embodiments, the cellulose substrate comprises a capture agent, optionally a capture agent comprising a CBD, immobilised thereon. In some embodiments, the kit further comprises a capture agent provided separately from the cellulose substrate. In some embodiments, the kit further comprises a reagent capable of producing a detectable signal upon reaction with the reporter agent. In various embodiments, the reporter agent, the capture agent and/or the analyte is soluble in the incubation solution/mixture/solvent.

In various embodiments, the amount of capture agent present in the incubation mixture and/or pre-immobilised on the cellulose substrate is not more than about 1000-fold molar excess, not more than about 900-fold molar excess, not more than about 800-fold molar excess, not more than about 700-fold molar excess, not more than about 600-fold molar excess, not more than about 500-fold molar excess, not more than about 400-fold molar excess, not more than about 300-fold molar excess, not more than about 200-fold molar excess, not more than about 100-fold molar excess, not more than about 90-fold molar excess, not more than about 80-fold molar excess, not more than about 70-fold molar excess, not more than about 60-fold molar excess, not more than about 50-fold molar excess, not more than about 40-fold molar excess, not more than about 30-fold molar excess, not more than about 20-fold molar excess, not more than about 10-fold molar excess, not more than about 9-fold molar excess, not more than about 8-fold molar excess, not more than about 7-fold molar excess, not more than about 6-fold molar excess or not more than about 5-fold molar excess of the analytes or of the typical amount range of analytes that is contained in a typical sample of a similar/identical type as the sample. In some embodiments, the amount of capture agent present in the incubation mixture and/or pre-immobilised on the cellulose substrate is not more than about 1000-fold molar excess, optionally not more than 100-fold molar excess, further optionally not more than 60-fold molar excess of the analyte. In various embodiments, the ratio of the moles of the capture agents to the moles of the analyte (or the typical molar range of analyte that is contained in a typical sample of a similar/identical type as the sample) in the incubation mixture is not more than about 1000, not more than about 900, not more than about 800, not more than about 700, not more than about 600, not more than about 500, not more than about 400, not more than about 300, not more than about 200, not more than about 100, not more than about 90, not more than about 80, not more than about 70, not more than about 60, not more than about 50, not more than about 40, not more than about 30, not more than about 20, not more than about 10, not more than about 9, not more than about 8, not more than about 7, not more than about 6 or not more than about 5.

The apparatus, method, system and kit disclosed herein may be employed in research settings to screen protein-protein interaction, and are not restricted to detection of immunocomplex formation. Embodiments of the apparatus, method, system and kit are also compatible with multi-channel pipette and plate reader, making it convenient to be adopted for lab usage. Embodiments of the apparatus, method, system and kit disclosed herein are compatible for use with samples like bodily fluid such as plasma, blood, urine and saliva apart from cell lysates, hence they may be easily applied in hospitals/clinic lab for diagnostic purposes where large number of samples can be processed. The apparatus, method and kit disclosed herein may be deployed for use as part of readily available commercial point-of-care diagnostic kits. By using cellulose-based vertical flow assaying method, embodiments of the apparatus, method, system and kit disclosed herein may be easily customized into a high-throughput format for lab-based diagnostic screening, saving substantial amounts of time, resources and reagents.

Accordingly, the apparatus, method, system and kit disclosed herein may be used for identifying a coronavirus infection, optionally a COVID-19 infection in a subject, by detecting the presence of a coronavirus protein, optionally SARS-CoV-2 protein, further optionally SARS-CoV-2 nucleocapsid protein or fragments thereof. Embodiments of the apparatus, method, system and kit may also be used for detecting neutralizing antibodies against a coronavirus infection, optionally a COVID-19 infection in a subject, for example, in a competitive binding assay format using SARS-CoV-2 Receptor Binding Domain (RBD) fused with CBD as the capture agent and labelled ACE2 as the reporter agent. As used herein, the term “identifying” as used herein in relation to a medical condition (such as an infection) is to be interpreted broadly to encompass determining a presence, an absence and/or a severity of the medical condition. The term “subject” as used herein includes patients and non-patients. The term “patient” refers to individuals suffering or are likely to suffer from a medical condition such as an infection, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

It will be appreciated that various embodiments discussed in different parts of the specification (e.g., under the different headers “apparatus/device”, “method”, “system/kit” etc) may be partially or fully combined, replaced, swapped etc with one another to form new embodiments.

### BRIEF DESCRIPTION OF FIGURES

FIG. 1 shows an apparatus for a cellulose-based vertical flow assay in accordance with an embodiment disclosed herein.

FIG. 2A shows the apparatus of FIG. 1 when the lid is engaged with the engaging members of the holder in the apparatus.

FIG. 2B shows the apparatus of FIG. 1 when the lid is disengaged from the engaging members of the holder in the apparatus.

FIG. 3A shows a test zone in the apparatus in accordance with an embodiment disclosed herein.

FIG. 3B shows the diameters of test zones in the apparatus in accordance with an embodiment disclosed herein.

FIGS. 4A, 4B, 4C and 4D show the front view, top view, side view and back view respectively of a lid of the apparatus in accordance with an embodiment disclosed herein.

FIGS. 5A, 5B, 5C, 5D and 5E show the front view, top view, side view, back view and perspective view respectively of a holder of the apparatus in accordance with an embodiment disclosed herein.

FIG. 6 is a schematic diagram of a method for detecting an analyte in a sample using embodiments of the apparatus as described herein.

FIG. 7 is a schematic diagram of another method for detecting an analyte in a sample using embodiments of the apparatus as described herein.

FIG. 8A and FIG. 8B show the results from validation experiments using embodiments of the apparatus and methods as described herein.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, mechanical, physical, chemical and biological changes may be made without deviating from the scope of the present disclosure. Example embodiments are not necessarily mutually exclusive as some may be combined with one or more embodiments to form new exemplary embodiments.

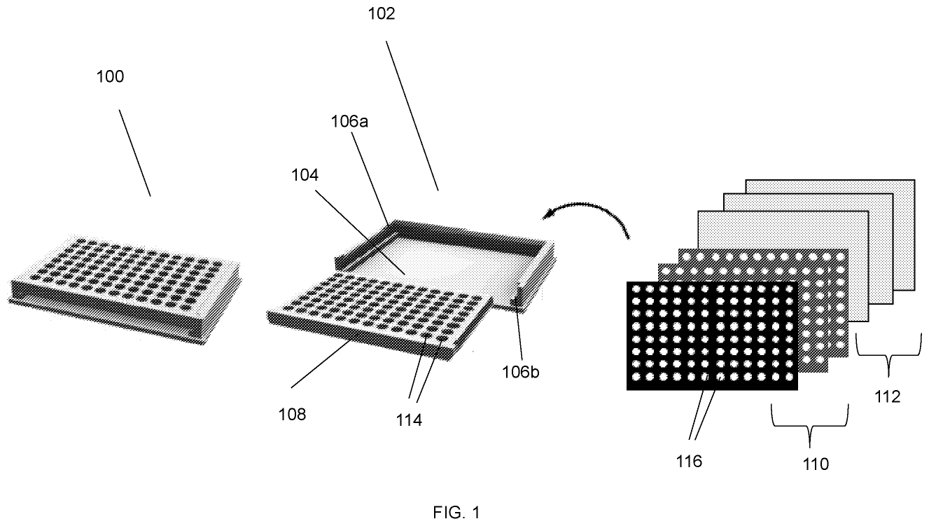

FIG. 1 shows an apparatus 100 for a cellulose-based vertical flow assay in accordance with an embodiment disclosed herein. The apparatus 100 comprises a holder 102 for supporting a cellulose substrate, the holder 102 having a base plate 104 and engaging members 106a and 106b, and a lid 108 for reversibly engaging with the engaging members 106a and 106b of the holder 102 in accordance with an embodiment disclosed herein. The apparatus 100 may further comprise one or more layers of cellulose substrate 110 and/or one or more layers of an absorbent material 112 disposed over the base plate 104 of the holder 102. The absorbent material 112 may be disposed between the cellulose substrate 110 and the base plate 104.

The cellulose substrate 110 may comprise a plurality of test zones 116. The lid 108 may comprise a plurality of openings 114 for allowing access to the test zones 116 on the cellulose substrate 110. In the example embodiment, the cellulose substrate 110 comprises 96 test zones 116 and the lid 108 comprises 96 openings 114 for accessing the 96 test zones 116. The number of test zones and openings may be adjusted i.e., increased or decreased, depending on the number of test zones and openings that are required. For example, a lid having 60 openings, 70 openings or 80 openings may be used together with a cellulose substrate having a matching number of test zones (i.e., 60 test zones, 70 test zones or 80 test zones).

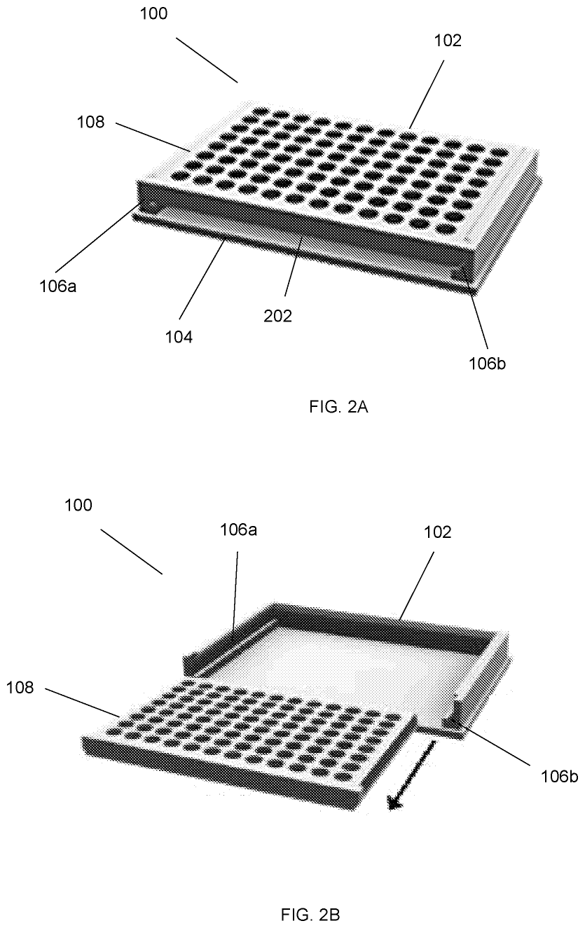

FIG. 2A shows the apparatus 100 when the lid 108 is engaged with the engaging members 106a and 106b of the holder 102. When engaged, the lid 108 is disposed over the base plate 104 and a gap 202 exists between the lid 108 and the base plate 104. In the example embodiment, the engaging members 106a and 106b are in the form of slots for slidably receiving/engaging the lid 108. The lid 108 is therefore slidably mounted on the holder 102 when engaged in the example embodiment. It will be appreciated that other suitable forms of engagement means between the holder 102 and the lid 108 may also be employed.

FIG. 2B shows the apparatus 100 when the lid 108 is disengaged from the engaging members 106a and 106b of the holder 102. The lid 108 is detachable from the holder 102 when disengaged in the example embodiment. In the example embodiment, the engaging members 106a and 106b are in the form of slots. The lid 108 may be disengaged from the engaging members 106a and 106b in the form of slots by a sliding motion. This detaches the lid 108 from the holder 102.

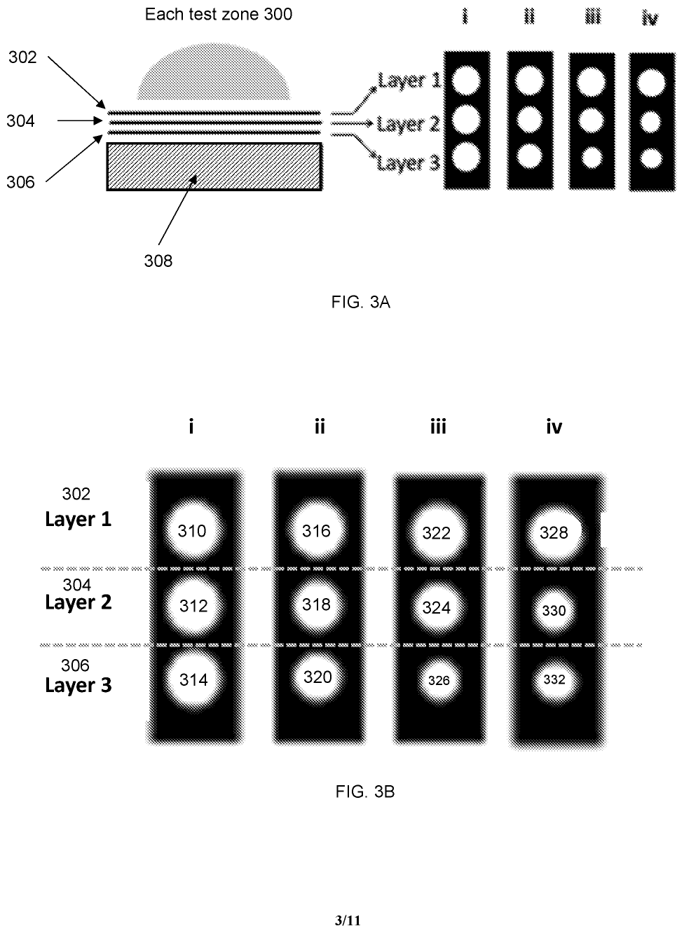

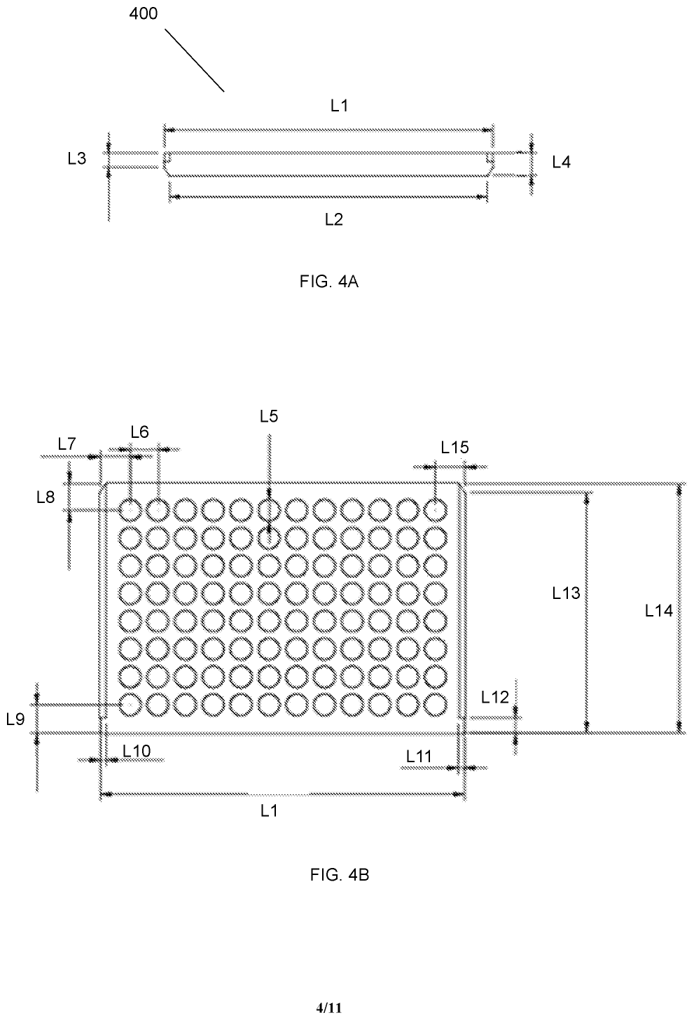

FIG. 3A shows a test zone 300 in accordance with an embodiment disclosed herein for receiving a sample to be tested. The test zone 300 may be substantially similar to the test zones 116 of FIG. 1. In the example embodiment, three cellulose substrates 302, 304 and 306 are stacked atop each other in a way such that the centre of each test zone in one cellulose substrate align with the centre of each test zone in the two other cellulose substrates along a vertical axis. The centre of each test zone in the three cellulose substrates 302, 304 and 306 may further align with an opening on the lid 108 of the apparatus 100 (not shown). The test zone 300 may be made more permeable to a liquid sample than areas of the cellulose substrate that are outside the test zone by coating the areas outside the test zone with wax (not shown). An absorbent material 308 may be placed below the cellulose substrates 302, 304 and 306 to absorb excess unbound molecules and/or provide a wicking/pulling force to enhance the flow of a sample through the test zone. Multiple layers of absorbent material may also be used to fit the gap of the apparatus.

FIG. 3B shows the diameters of test zones on the three cellulose substrates 302, 304 and 306 in FIG. 3A in four different scenarios (i) to (iv). 310, 316, 322 and 328 respectively represent the test zones on the first layer of cellulose substrate 302 for each of the four different scenarios (i) to (iv). 312, 318, 324 and 330 respectively represent the test zones on the second/middle layer of cellulose substrate 304 for each of the four different scenarios (i) to (iv). 314, 320, 326 and 332 respectively represent the test zones on the third/final layer of cellulose substrate 306 for each of the four different scenarios (i) to (iv). Varying the diameters of the test zones across the different layers varies a flow rate of the sample applied. This is illustrated in the four scenarios (i) to (iv) (e.g., see layers 2 and 3, i.e., cellulose substrates 304 and 306). In scenario (i), when the diameters of the test zones 310, 312 and 314 in cellulose substrates 302, 304 and 306 are [5 mm, 5 mm, 5 mm] (See scenario (i) in FIG. 3B comprising a combination of test zones 310, 312 and 314 across the three layers of cellulose substrates 302, 304 and 306) respectively, a high flow rate is achieved. In scenario (iv), when the diameters of the test zones 328, 330 and 332 in cellulose substrates 302, 304 and 306 are [5 mm, 3 mm, 3 mm] (See scenario (iv) in FIG. 3B comprising a combination of test zones 328, 330 and 332 across the three layers of cellulose substrates 302, 304 and 306) respectively, a low flow rate is achieved. In scenarios (ii) and (iii), moderate flow rate is achieved when the diameters of the test zones 316, 318 and 320 in cellulose substrates 302, 304 and 306 are [5 mm, 4 mm, 4 mm](See scenario (ii) in FIG. 3B comprising a combination of test zones 316, 318 and 320 across the three layers of cellulose substrates 302, 304 and 306) respectively and [5 mm, 4 mm, 3 mm] respectively (See scenario (iii) in FIG. 3B comprising a combination of test zones 322, 324 and 326 across the three layers of cellulose substrates 302, 304 and 306). The flow rate decreases from scenarios (i) to (iv). It will be appreciated that the diameters of the test zones are not limited to those shown of scenarios (i), (ii), (iii) and (iv) as shown in this figure and they may be further varied to achieve the desired flow rate. For example, it will be appreciated that where a plurality of test zones is present on a layer of substrate, these test zones may have the same or different diameters. When they have different diameters, the different scenarios like those depicted in scenarios (i) to (iv) may be carried out simultaneously on a single platform (e.g., test zones 312, 318, 324 and 330 may all be present simultaneously on a single cellulose substrate making up a second/middle layer and 314, 320, 326 and 332 may all be present simultaneously on a single cellulose substrate making up a third/final layer in an assay).

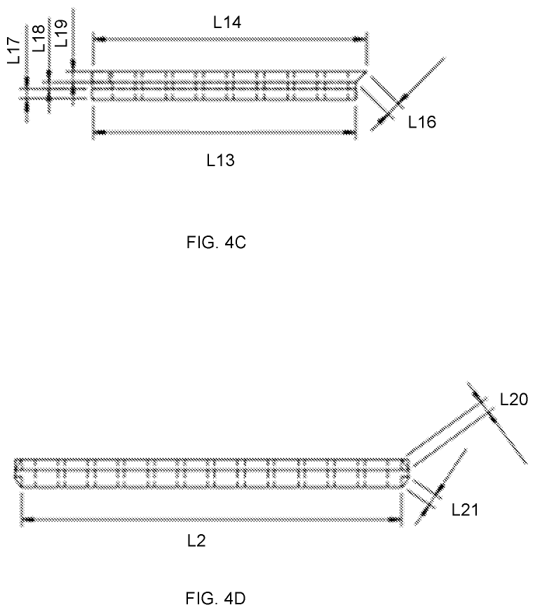

FIGS. 4A, 4B, 4C and 4D show the front view, top view, side view and back view respectively of a lid 400 in accordance with an embodiment disclosed herein. The lid 400 may be substantially similar to the lid 108 of FIG. 1. In the example embodiment, the dimensions of the lid 400 are as follows: L1=118.26-118.27 mm, L2=114-114.36 mm, L3=5.07 mm, L4=8.02 mm, L5=6.9 mm, L6=9 mm, L7=9.82 mm, L8=8.74 mm, L9=9.26 mm, L10=1.97 mm, L11=1.95 mm, L12=5 mm, L13=78.01 mm, L14=81 mm, L15=9.5 mm, L16=4.23 mm, L17=2.95 mm, L18=2.05 mm, L19=3.02 mm, L20=3.67 mm and L21=3.6 mm. It will be appreciated that the dimensions of a lid are not limited to those mentioned above and may be further varied.

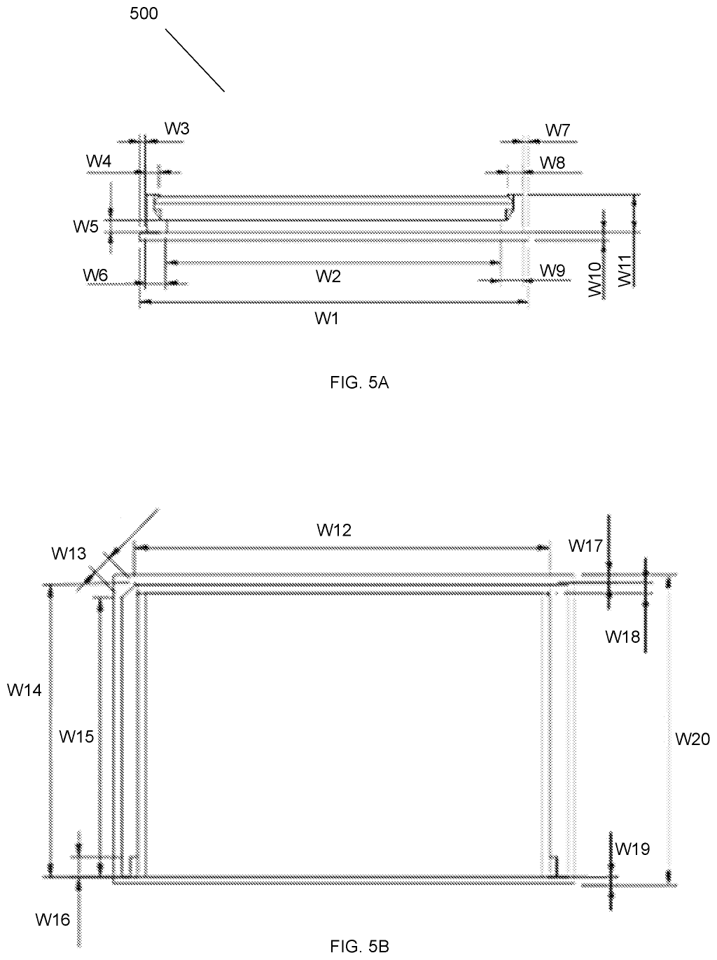

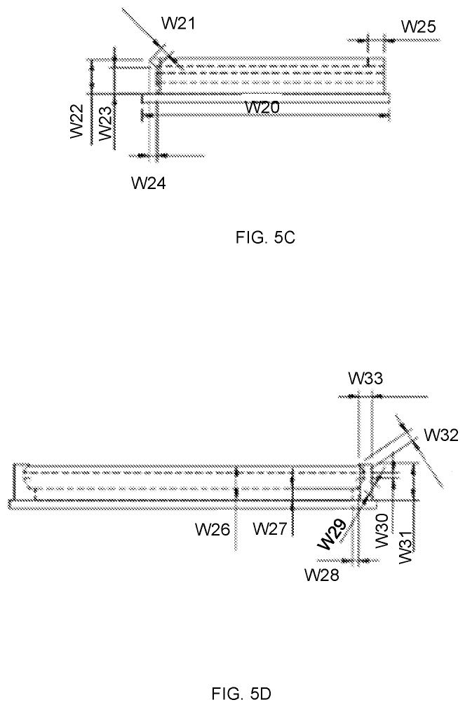

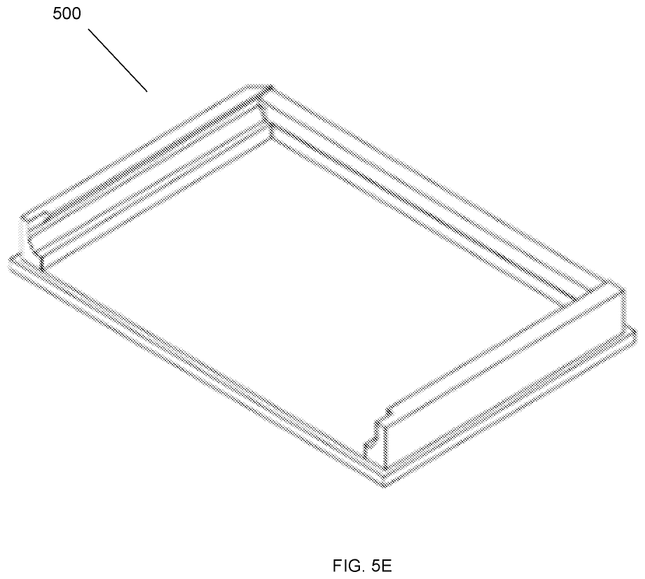

FIGS. 5A, 5B, 5C, 5D and 5E show the front view, top view, side view, back view and perspective view respectively of a holder 500 in accordance with an embodiment disclosed herein. The holder 500 may be substantially similar to the holder 102 of FIG. 1. In the example embodiment, the dimensions of the holder 500 are as follows: W1=127.8 mm, W2=110 mm, W3=2 mm, W4=4.43 mm, W5=4 mm, W6=6.73 mm, W7=1.8 mm, W8=4.97 mm, W9=7.27 mm, W10=2.98 mm, W11=12.52 mm, W12=115.06 mm, W13=5.47 mm, W14=81 mm, W15=77.13 mm, W16=5.52 mm, W17=2.5 mm, W18=2.5 mm, W19=2 mm, W20=85.5 mm, W21=3.55 mm, W22=11.81 mm, W23=9.29 mm, W24=2.5 mm, W25=5.52 mm, W26=11.81 mm, W27=4 mm, W28=2 mm, W29=3.98 mm, W30=2.06 mm, W31=12.52 mm, W32=3.53 mm and W33=4.43 mm. It will be appreciated that the dimensions of a holder are not limited to those mentioned above and may be further varied.

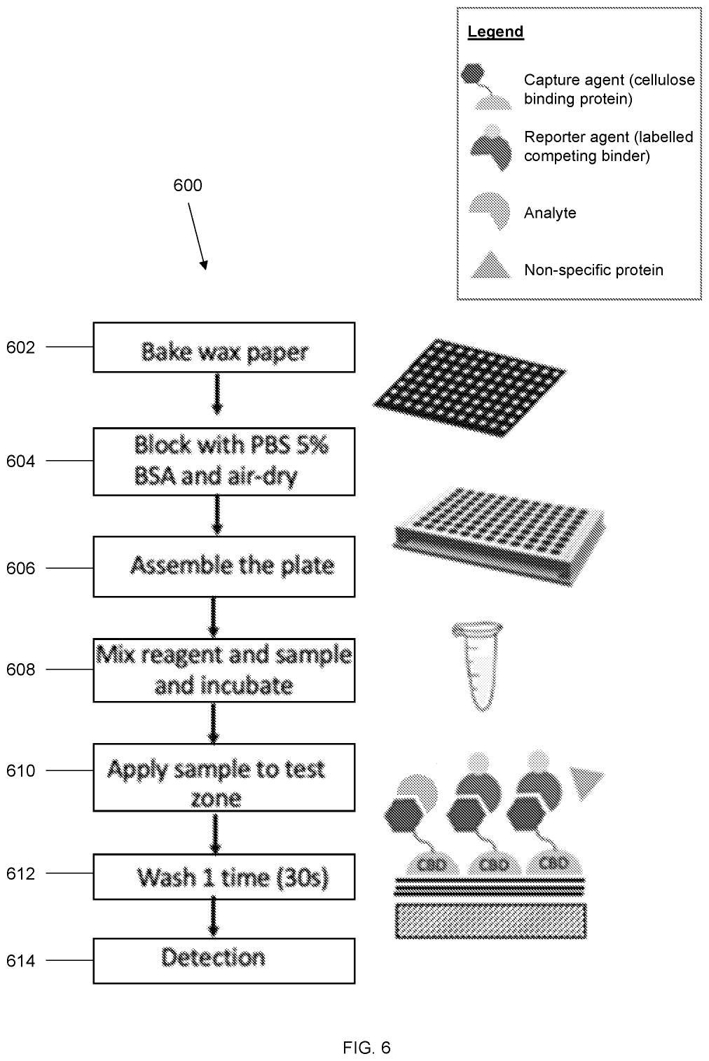

FIG. 6 is a schematic diagram of a method 600 for detecting an analyte in a sample using embodiments of the apparatus as described herein. Method 600 described herein is a pre-mix method where the reagents and samples are mixed in the solution and incubated to allow complex formation before dispensing on the test zones. To prepare the substrate, a waxed cellulose substrate is first baked at about 150° C. for about 1 minute before it is blocked with phosphate buffered saline (PBS) and 5% bovine serum albumin (BSA) and then air-dried in steps 602 and 604. Next, in step 606, the apparatus is assembled. One or more layers of an absorbent material (e.g., Whatman GB005 blotting papers) together with one or more layers of waxed cellulose substrate stacked atop the absorbent material are aligned along a side of the lid such that the centers of the 96 test zones on the cellulose substrate will be substantially aligned with the centers of the 96 holes defined by the lid when the lid is in a fully engaged configuration. The lid is then slidably inserted into the holder together with the absorbent material and the cellulose substrate so that the lid is mounted on the holder with the absorbent material and waxed cellulose substrate disposed in the gap between the lid and the holder. Alternatively, the one or more layers of an absorbent material may be disposed on the base plate of the holder first, followed by the one or more layers of waxed cellulose substrate. The lid is then slidably inserted into the holder so that the lid is mounted on the holder with the absorbent material and waxed cellulose substrate disposed in the gap between the lid and the holder. In step 608, the samples are mixed in solution with a reporter agent and a capture agent in the form of cellulose binding proteins/aptamers comprising a cellulose binding domain and incubated to allow complex formation (e.g., formation of reporter agent-analyte-capture agent complex in the case of a non-competitive binding assay (not shown) or formation of reporter agent-capture agent complex and analyte-capture agent complex in the case of a competitive-binding assay as shown in the figure) before dispensing on test zones in step 610. The reaction time for sample is dependent on the association (kₒₙ) and dissociation rate (k_{off}) of the protein-ligand of interest. For example, protein-ligand interaction with fast kₒₙ and slow k_{off} will require less incubation time for pre-mix method while protein-ligand interaction with slow kₒₙ and fast k_{off} will require more incubation time. After the reagents are fully absorbed by the test zones, the test zones are then washed with a washing reagent e.g., once for about 30 s to remove free molecules from the cellulose substrate and thereby remove noise in the subsequent detection step 614. Detection may be carried out by detecting whether a signal is effected by the reporter agent using a microtiter plate reader. In a non-competitive binding assay, the presence of a signal indicates that the analyte is present in the sample and is captured on the cellulose substrate by the capture agent, and the absence of a signal indicates that the analyte is not present in the sample. A pre-wet test zone comprising sample only or with wash reagent can be used as a control for baseline subtraction. In a competitive binding assay, the presence of a signal may indicate that little or no analyte is present in the sample and captured on the cellulose substrate by the capture agent.

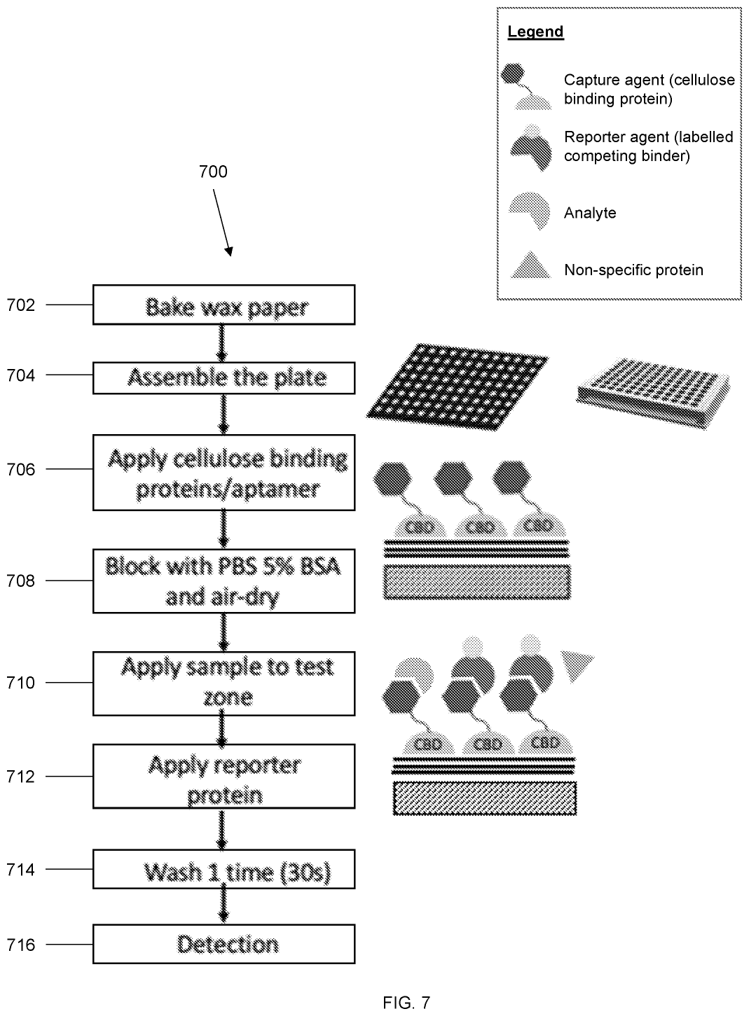

FIG. 7 is a schematic diagram of a method 700 for detecting an analyte in a sample using embodiments of the apparatus as described herein. In the example embodiment, the method 700 is a pre-immobilisation method that comprises first coating a capture agent on the cellulose substrate, followed by application of sample, reporter agent and wash reagent sequentially before detection. To prepare the substrate, a waxed cellulose substrate is first baked at about 150° C. for about 1 minute in step 702. Next, in step 704, the apparatus is assembled. One or more layers of an absorbent material (e.g., Whatman GB005 blotting papers) together with one or more layers of waxed cellulose substrate stacked atop the absorbent material are aligned along a side of the lid such that the centers of the 96 test zones on the cellulose substrate will be substantially aligned with the centers of the 96 holes defined by the lid when the lid is in a fully engaged configuration. The lid is then slidably inserted into the holder together with the absorbent material and the cellulose substrate so that the lid is mounted on the holder with the absorbent material and waxed cellulose substrate disposed in the gap between the lid and the holder. Alternatively, the one or more layers of an absorbent material may be disposed on the base plate of the holder first, followed by the one or more layers of waxed cellulose substrate. The lid is then slidably inserted into the holder so that the lid is mounted on the holder with the absorbent material and waxed cellulose substrate disposed in the gap between the lid and the holder. A capture agent in the form of a cellulose binding protein/aptamer comprising a cellulose-binding domain is applied to the test zone in step 706 to immobilise the capture agent on the test zones. The test zones are then blocked with PBS and 5% BSA, and then air-dried in step 708. A sample to be tested is applied to the test zone in step 710 to allow the analyte, if present in the sample, to bind to the capture protein immobilised on the test zones. The reaction time for sample is dependent on the association (kₒₙ) and dissociation rate (k_{off}) of the protein-ligand of interest. For example, protein-ligand interaction with fast kₒₙ and slow k_{off} will require a shorter reaction time while protein-ligand interaction with slow kₒₙ and fast k_{off} will require a longer reaction time. In the case of pre-immobilization method, the flow rate can be adjusted, for example, by varying the diameters of the test zones across the substrates, for shorter or longer encounter time with capture protein for optimal signal detection depending on the kinetics. A washing step may be carried out after the sample is fully absorbed by the test zones. A reporter agent in the form of a labelled analyte binder (not shown) is then applied to the test zones in step 712 to bind to any analyte captured by capture agent on the cellulose substrate in the case of a non-competitive binding assay. In the case of a competitive binding assay, a reporter agent in the form of a labelled competing binder is applied to the test zones to compete with the analyte for binding to the capture agent on the cellulose substrate as shown in the figure. The test zones are then washed with a washing reagent, e.g., once for about 30 seconds, in step 714 to remove free molecules from the cellulose substrate and thereby remove noise in the subsequent detection step 716. Detection may be carried out by detecting whether a signal is effected by the reporter agent using a microtiter plate reader. In a non-competitive binding assay, the presence of a signal indicates that the analyte is present in the sample and is captured on the cellulose substrate by the capture agent, and the absence of a signal indicates that the analyte is not present in the sample. A pre-wet test zone comprising sample only or with wash reagent can be used as a control for baseline subtraction. In a competitive binding assay, the presence of a signal may indicate that little or no analyte is present in the sample and captured on the cellulose substrate by the capture agent.

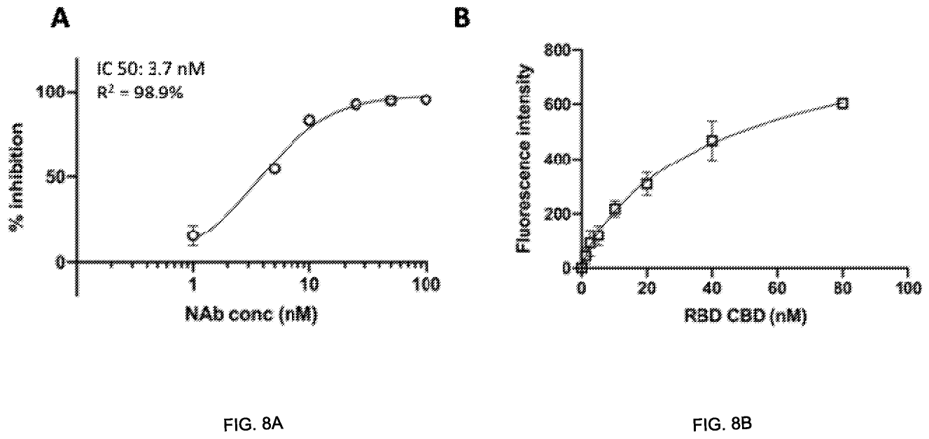

FIG. 8A and FIG. 8B show the results from validation experiments using embodiments of the apparatus and methods as described herein. FIG. 8A shows the inhibition of capture protein by neutralizing antibody detected in blood plasma matrix when competing against its ligand, ACE2. FIG. 8B shows the fluorescence intensity of ACE2-RBD-CBD complex formation in whole blood sample when measured against increasing concentration of RBD-CBD (capture protein) coated on the cellulose substrate. In this example, two competitive ELISA vertical flow experiments were performed to validate the setup. SARS-CoV-2 Receptor Binding Domain (RBD) binds to human Angiotensin Converting Enzyme-2 (ACE2) protein on the cell surface as the first contact to elicit infection in human cells. As a defence mechanism, the immune system responds by producing neutralizing antibody (NAb) to block the complex formation of RBD-CBD and ACE2 to prevent viral invasion. The pre-mix method (see e.g., FIG. 6) was employed to measure the effect of NAb competition against ACE2 on RBD protein. First, RBD protein fused with CBD (RBD-CBD) was recombinantly expressed and ACE2 protein was labelled with Alexafluor594 as reporter agent. Plasma spiked with mouse monoclonal SARS-CoV-2 neutralizing antibody at a concentration range of 1, 5, 10, 25, 50 and 100 nM were incubated with 2.5 nM of RBD-CBD and 2.5 nM fluorescent conjugated ACE2 protein in 2:1:1 volume ratio for 5 minutes. A total of 40 μL sample volume was dispensed on the 5 mm test zone, followed by a 40 μL of Phosphate Buffer Saline (PBS) as a washing step. Then, the plate was placed in the TECAN Microplate reader Infinite® 200 PRO for signal detection. A negative control was performed using plasma without NAb to measure the maximum signal. The baseline of fluorescence signal was measured without RBD-CBD in the premix sample. The assay was able to detect 50% inhibition of ACE2-RBD binding at 3.7 nM of neutralizing antibody (FIG. 8A). Using the same protein combination, the assay was able to detect RBD-CBD and fluorescence conjugated ACE2 complex formation in blood sample when RBD-CBD is pre-immobilized on the cellulose paper (FIG. 8B).

It will be appreciated by a person skilled in the art that other variations and/or modifications may be made to the embodiments disclosed herein without departing from the spirit or scope of the disclosure as broadly described. For example, in the description herein, features of different exemplary embodiments may be partially or fully mixed, combined, interchanged, incorporated, adopted, modified, included etc. or the like across different exemplary embodiments. The present embodiments are, therefore, to be considered in all respects to be illustrative and not restrictive.

1. An apparatus for a cellulose-based vertical flow assay, the apparatus comprising:
a holder for supporting a cellulose substrate having a plurality of test zones thereon, the holder having a base plate and engaging members; and
a lid for reversibly engaging with the engaging members of the holder such that when engaged, the lid is disposed over the base plate and a gap exists between the lid and the base plate,
wherein the lid comprises a plurality of openings for allowing access to the test zones on the cellulose substrate.

2. The apparatus according to claim 1, wherein the apparatus further comprises one or more layers of cellulose substrate disposed over the base plate of the holder, and the cellulose substrate having a plurality of test zones thereon.

3. The apparatus according to claim 1, wherein the apparatus further comprises one or more layers of an absorbent material disposed over the base plate, and optionally wherein the absorbent material is disposed between a cellulose substrate and the base plate.

4. The apparatus according to claim 1, wherein the gap is capable of accommodating a plurality of layers of a cellulose substrate and a plurality of layers of an absorbent material simultaneously.

5. The apparatus according to claim 1, wherein the engaging members of the holder comprise slots for slidably engaging the lid.

6. The apparatus according to claim 1, wherein the plurality of openings of the lid each aligns to a respective test zone of the cellulose substrate.

7. The apparatus according to claim 1, wherein the test zones of the cellulose substrate are relatively more permeable to a liquid sample than areas of the cellulose substrate that are outside the test zones, and optionally wherein the areas of the cellulose substrate that are outside the test zones are coated with a layer that is substantially impermeable to a liquid sample.

8. The apparatus according to claim 1, wherein the lid comprises no less than 60 openings for allowing access to the test zones on the cellulose substrate.

9. The apparatus according to claim 2, wherein the one or more layers of cellulose substrate comprises a plurality of layers of cellulose substrate and wherein the diameter of the test zones of a cellulose substrate selected from the plurality of layers of cellulose substrate is different from the diameter of the test zones of another cellulose substrate selected from the plurality of layers of cellulose substrate.

10. A method of detecting an analyte in a sample, the method comprising:
providing an apparatus comprising, a holder having a base plate and engaging members; one or more layers of cellulose substrate disposed over the base plate, the cellulose substrate having a plurality of test zones thereon; one or more layers of an absorbent material disposed between the base plate and the cellulose substrate; and a lid reversibly engaged with the engaging members of the holder such that the lid is disposed over the base plate, the cellulose substrate and the absorbent material, and wherein the lid comprises a plurality of openings for allowing access to the test zones on the cellulose substrate;
applying the sample to one or more test zones of the cellulose substrate through the one or more openings of the lid to allow said analyte, if present in said sample, to be captured onto said one or more test zones of the cellulose substrate by a capture agent that is immobilized on the one or more test zones, wherein said capture agent is:
(i) incubated with said sample prior to the applying step; and/or
(ii) immobilised on the one or more test zones of the cellulose substrate prior to the applying step; and
determining a presence or absence of said analyte captured on the one or more test zones of said cellulose substrate by said capture agent.

11. The method according to claim 10, wherein determining a presence or absence of said analyte captured on the one or more test zones by said capture agent comprises detecting a signal effected by a reporter agent on the one or more test zones, wherein said reporter agent:
(iii) comprises an analyte binder and said reporter agent is contacted with the sample to allow said reporter agent to bind to said analyte, if present, in said sample; or
(iv) comprises a competing binder having affinity for said capture agent and said reporter agent is contacted with said capture agent to allow said reporter agent to bind to said capture agent.

12. The method according to claim 10, wherein the method further comprises, after the applying step but prior to the determining step, washing the one or more test zones with a washing reagent to remove free molecules from the cellulose substrate.

13. The method according to claim 10, wherein the method further comprises blocking the cellulose substrate with a blocking agent.

14. The method according to claim 11, wherein where said reporter agent comprises an analyte binder, the method comprises incubating the reporter agent and the capture agent with said sample prior to the applying step and said analyte when present, is bound to the reporter agent to form part of a reporter agent-analyte-capture agent complex prior to the applying step.

15. The method according to claim 11, wherein where said reporter agent comprises an analyte binder, the method comprises incubating the reporter agent with said sample and immobilizing said capture agent on the one or more test zones of said cellulose substrate prior to the applying step and said analyte when present, is bound to the reporter agent to form a reporter agent-analyte complex prior to the applying step.

16. The method according to claim 11, wherein where said reporter agent comprises a competing binder, the method comprises incubating said capture agent with said reporter agent and/or said sample prior to the applying step to form a reporter agent-capture agent complex and/or an analyte-capture agent complex prior to the applying step.

17. The method according to claim 11, wherein where said reporter agent comprises a competing binder, the method comprises immobilizing said capture agent on the one or more test zones of said cellulose substrate prior to the applying step and dispensing the reporter agent on the one or more test zones after the applying step.

18. The method according to claim 10, wherein the applying step comprises allowing the sample to flow through the one or more test zones of the cellulose substrate to the absorbent material.

19. The method according to claim 11, further comprising adding a reagent capable of producing a detectable signal upon reaction with the reporter agent after the applying step.

20. The method according to claim 10, wherein the capture agent has affinity for cellulose, and optionally wherein the capture agent comprises a cellulose binding domain.
